SEQUENCE LISTING (2 sequences)

SEQ ID NO: 1 - RNA, 21 nt, Artificial Sequence
  shRNA sequence inhibiting ITGA7 expression
aaagguagcaaaucccggagg

SEQ ID NO: 2 - RNA, 21 nt, Artificial Sequence
  shRNA sequence inhibiting ITGA7 expression
aucuaacacauaguccagggc

(19) United States
(12) Patent Application Publication
(10) Pub. No.: US 2015/0315276 A1
(43) Pub. Date: Nov. 5, 2015

(54) INHIBITOR OF INTEGRIN FOR THE TREATMENT OR PREVENTION OF TUMOURS

(71) Applicant: ISTITUTI FISIOTERAPICI OSPITALIERI (IFO) -ISTITUTO REGINA ELENA PER LO STUDIO E LA CURA DEI TUMORI, Roma (IT)

(72) Inventors: Ruggero DE MARIA MARCHIANO, Roma (IT); Tobias Longin HAAS, Roma (IT); Lucia RICCI VITIANI, Roma (IT); Mauro BIFFONI, Roma (IT)

(21) Appl. No.: 14/441,837

(22) PCT Filed: Nov. 8, 2013
(86) PCT No.: PCT/IB13/59993

§ 371 Date: May 9, 2015

(30) Foreign Application Priority Data
Nov. 9, 2012 (IT) RM2012A000550

(51) Int. Cl. C07K 16/28; C12N 15/113

(52) U.S. Cl. CPC C07K 16/2842; C12N 15/1138; A61K 2039/505

(57) ABSTRACT

The present invention relates to inhibitors of alpha7 integrin for the treatment, suppression and/or prevention of tumours, to composition comprising inhibitors of alpha7 integrin for the treatment, suppression and/or prevention of tumours and to medical treatments comprising administering said inhibitors or compositions to a patient in need thereof, for the treatment, suppression and/or prevention of tumours.

### FIELD OF THE INVENTION

The present invention relates to the field of tumour therapy, in particular to the identification of new molecules for tumour suppression, therapy and/or prevention, to compositions comprising the same and to medical treatments comprising administration of said compounds for the treatment, suppression and/or prevention of tumours.

Furthermore, the present invention relates to inhibitors of alpha7 integrin for the treatment, suppression and/or prevention of tumours, to composition comprising inhibitors of alpha7 integrin for the treatment, suppression and/or prevention of tumours and to medical treatments comprising administering said inhibitors or compositions to a patient in need thereof, for the treatment, suppression and/or prevention of tumours.

### STATE OF THE ART

In recent years great progress has been made in the field of neutralizing integrin function as new form of targeted therapy in cancer. Preclinical and also clinical studies on molecules inhibiting specific integrins function have shown to be effective in blocking tumour growth. The integrin v 3 and v 5 inhibitor cilengitide, for example, has already completed phase II clinical trials in the treatment of patients with glioblastoma multiforme (Reardon, D. A. et al. Randomized phase II study of cilengitide, an integrin-targeting arginine-glycine-aspartic acid peptide, in recurrent glioblastoma multiforme. J Clin Oncol 26, 5610-5617 (2008)). Currently the recruitment for phase III studies (CENTRIC trail) is completed and the study is on-going. While cilengitide is a small peptide inhibitor blocking the integrin attachment mimicking the binding side of the natural ligand fibronectin, there are also anti integrin antibodies in preclinical and clinical trials. MEDI-522 blocks v 3 function, while CNTO95 blocks both, integrin v 3 and v 5. Like cilengitide, both antibodies inhibit angiogenisis, and demonstrated a certain degree of efficacy in phase I or phase II clinical trials, respectively. Also an antibody targeting alpha5beta1 integrin is currently in clinical trials. Volociximab proved to be well tolerated by the patients and is currently in phase II trials for the treatment of solid tumours. All of the integrin inhibitors developed to date have in common, that they inhibit integrins that do not directly interfere with tumour cell growth, but inhibit mainly tumour vascularization, thus acting mainly on the tumour microenvironment.

In connection to the above, it has to be noted that the therapeutic value of anti-vascular drugs in highly aggressive tumours such as e.g. glioblastoma multiforme and others, is currently highly debated. One example is the humanized monoclonal antibody bevacizumab. Although it is FDA approved for recurrent GBM, its therapeutic efficacy is mild and might, at least partially, be rather based on secondary effects (Gonzalez, J., Kumar, A. J., Conrad, C. A. & Levin, V. A. Effect of bevacizumab on radiation necrosis of the brain. International journal of radiation oncology, biology, physics 67, 323-326 (2007) and Wong, E. T., Huberman, M., Lu, X. Q. & Mahadevan, A. Bevacizumab reverses cerebral radiation necrosis. J Clin Oncol 26, 5649-5650 (2008)).

The lack of highly functional therapeutic options to inhibit integrin mediated tumour progression underlines the importance to define new molecules and pathways active in malignant cells.

In the scientific paper published by J H Luo and coworkers in 2007 (Analysis of integrin alpha7 mutations in prostate cancer, liver cancer, glioblastoma multiforme, and leiomyosarcoma. Ren B, Yu Y P, Tseng GC, Wu C, Chen K, Rao U N, Nelson J, Michalopoulos G K, Luo J H. J Natl Cancer Inst. 2007 Jun. 6; 99(11):868-80) it is stated that the ITGA7 gene is mutated in a high percentage of Prostate Cancer, Liver Cancer, Glioblastoma Multiforme,and Leiomyosarcoma. In this and two follow up papers (Han, Y. C. et al. Interaction of integrin-linked kinase and miniature chromosome maintenance 7-mediating integrin {alpha}7 induced cell growth suppression. Cancer research 2010 70, 4375-43849, and Zhu, Z. H. et al. Integrin alpha 7 interacts with high temperature requirement A2 (HtrA2) to induce prostate cancer cell death. The American journal of pathology 2010; 177(3):1176-86) the authors claim that ITGA7 has an important role as tumour-suppressor in lung and prostate cancer models.

In 2010 Justin D. Lathia et al. (Lathia, J. D. et al. Integrin alpha 6 regulates glioblastoma stem cells. 2010 Cell stem cell 6, 421-432) published a paper describing integrin alpha6beta1 as essential for the growth of primary glioblastoma stem cell-like cells. However, the phenotype of the knockout mice was strongly discouraging for a possible therapeutic targeting of the integrin alpha6, as it is described in the art (Georges-Labouesse, E. et al. Absence of integrin alpha 6 leads to epidermolysis bullosa and neonatal death in mice. Nature genetics 13, 370-373 (1996)) that 100% of embryos die early in development, when ITGA6 is deleted.

Also the beta1 subunit is published to support tumour growth in other model systems such as lung cancer and lymphoma (Morello, V. et al. beta1 integrin controls EGFR signaling and tumourigenic properties of lung cancer cells. 2011, Oncogene 30, 4087-4096 and Stroeken, P. J., van Rijthoven, E. A., van der Valk, M. A. & Roos, E. Targeted disruption of the beta1 integrin gene in a lymphoma cell line greatly reduces metastatic capacity. Cancer research 58, 1569-1577 (1998)). However, interference with integrin beta1 function either by using blocking antibodies, or genetic interference might also result in incalculable side effects, as integrin beta1 forms heterodimers with several alpha subunits. This is also supported by severe phenotypes of complete and tissue specific genetic ablations of the ITGB1 gene (Bombardelli, L. et al. Pancreas-specific ablation of beta1 integrin induces tissue degeneration by disrupting acinar cell polarity. Gastroenterology 138, 2531-2540, 2540 e2531-2534; Fassler, R. & Meyer, M. Consequences of lack of beta 1 integrin gene expression in mice. Genes & development 9, 1896-1908 (1995); Fassler, R. et al. Differentiation and integrity of cardiac muscle cells are impaired in the absence of beta 1 integrin. Journal of cell science 109 (Pt 13), 2989-2999 (1996); Stephens, L. E. et al. Deletion of beta 1 integrins in mice results in inner cell mass failure and peri-implantation lethality. Genes & development 9, 1883-1895 (1995)).

The patients' benefit from current therapeutic treatment of the large majority of solid tumours is still very limited. In most cases these treatments prolong the life-span and can lead to an increase in quality of life. However, for some very aggressive tumours such as GBM (Glioblastoma multiforme), curative treatments are far from being realized and the current medication might also bear big potential complications for the patient. This led to the generation of “smarter” drugs, targeting specifically pathways employed mainly by the tumour cells or within the tumour microenvironment. One example is the interference with tumour-induced neovascularization for example by the treatment with novel integrin inhibition molecules. However these treatments are also not free of risks. Complications of these innovative, angiogenesis targeting treatments could be that the tumour cells evade their hypoxic environment, forming after the therapeutic treatment and infiltrate deeper into the surrounding tissue. Indeed it was experimentally shown, that anti-angiogenic treatments with bevacizumab leads to an increase of tumour cell infiltration in a rat xenograft model of glioblastoma multiforme. This can also explain that low dose treatment with this drug has apparently a better clinical outcome, when compared to higher doses. In summary the current therapeutic options to treat highly aggressive tumours are in progress, but it is getting evident, that new, innovative treatment options are indispensable. According to the present state of the art, hence, the problem of identifying new targets for tumour suppression, therapy or prevention is still opened, in particular, targets for tumour suppression, therapy or prevention in early stages of the tumour or capable of reduce the aggressiveness of tumours are highly desirable.

### SUMMARY OF THE INVENTION

The present inventor have surprisingly found and demonstrated that inhibition of integrin alpha7 function has a profound anti-tumour activity. The data reported in the experimental section below demonstrate that integrin alpha 7 is expressed on stem-like tumour cells that are particularly aggressive and tumourigenic. The experiments carried out by the inventors demonstrate that inhibition of the integrin alpha 7 activity either by inhibition of the expression of the protein or by inhibition by direct interaction with the protein expressed on the cell surface, result in a strong anti-tumour activity.

The data obtained by the present inventors and reported in the experimental section below, indicate that at least integrin alpha7 (probably in conjunction with integrin beta1 as heterodimer), is expressed by all primary tumour stem cell-like cell lines investigated. The expression in most of the lines is very high, especially when compared to commercially available cell lines.

These data are in contradiction with the finding published by Luo and coworkers 2007 discussed in the prior art section above.

The main difference between the experiments carried out by the inventors and the study performed by Luo and coworkers discussed in the prior art section, lies in the cellular system used. While the results of the present inventors were entirely obtained on primary brain tumour stem-like cells, Luo and coworkers used long-term established differentiated prostate and lung cancer cell lines. It is broadly accepted, that cells massively change their behaviour under standard cell culture conditions. These changes can be also observed, but are much less pronounced under the spheroid culture conditions that were applied by the present inventors to the stem-like cells. The spheroids represent a 3-dimensional tumour growth system, which better mimics the situation in the patient when compared to standard 2D cultures. In addition, it is very likely that adherent cell lines completely change their dependency on different cell adhesion molecules due to their need to attach to the plastic in the culture dish.

The inventors could indeed show that tumour cells (such as glioblastoma, neuroblastoma and other tumour cells), kept under normal cell culture display a low expression of integrin alpha7 surface expression, when compared to spheroid cultures of cells deriving from the same tumours, suggesting a down regulation of this molecule under these conditions.

Another advantage the system used by the inventors is the comparable low passage number of our cells used in the experiments (5 to max. 30 passages, depending on the line used). All these points, also supported by in vivo results in mice discussed in the experimental section below, suggest that the spheroid BTSC model system used by the inventors is a better model of primary tumour when compared to the standard 2D cell culture system used in the art.

Furthermore, the preclinical in vivo data obtained in the art, were obtained with clonal lines ectopically overexpressing the ITGA7 cDNA. These cells were shown to be less tumourigenic when compared to the control cells. However overexpression experiments bear a high risk for artifacts due to the alteration of the equilibrium of the proteins expressed. Especially the overexpression of an integrin alpha subunit such as ITGA7 bears this risk. It is well known, that the beta1 subunit has to form a heterodimer with most alpha subunits for the proper function of the integrin. In an ectopic expression scenario the beta1 subunit is likely to be sequestered by the overexpressed ITGA7, leading to an change of the surface expression and/or function of other alpha integrins.

Hence, in contrast with the previously published works, the present application discloses that on cellular models that are much more similar to in vivo situations as well as in in vivo experiments on mice, the inhibition of the integrin alpha 7 activity produces a strong anti-tumour effect.

Objects of the invention are hence: an inhibitor of integrin alpha 7 activity for use in the treatment and/or prevention of tumours, compositions comprising said inhibitor and a pharmaceutical acceptable carrier for use in the treatment and/or prevention of tumours and a method for the treatment and/or prevention of tumours comprising administering an effective amount of an inhibitor of integrin alpha 7 activity or of a composition comprising the same to a patient in need thereof.

### GLOSSARY

According to the present invention, “an inhibitor of integrin alpha 7 activity” can be an inhibitor acting at any biological level, hence the inhibitor can either interfere with the expression of the protein, e.g. by inhibiting the transcription of the integrin alpha 7 gene or by inhibiting the translation of the integrin alpha 7 RNA or it can interfere with the activity of the integrin alpha 7 protein (ITGA7) on the cell surface e.g. by inhibiting the formation of the ITGA7-ITGB1 heterodimer or the binding of ITGA7 or of the heterodimer with ligands that activate the complex, such as laminin, or by blocking the activation of the focal adhesion kinase (FAK) by the ITGA7-ITGB1 heterodimer and the like. In principle, the phrase “an inhibitor of integrin alpha 7 activity” comprises indirect inhibitors, e.g. any molecule or group of molecules that can impair (inhibit) the expression of the ITGA7 protein, and direct inhibitors e.g. any molecule or group of molecules that can impair the activity of the ITGA7 protein, also in its heterodimeric form, on the cell surface.

According to the present invention, the phrase “RNA-based inhibitor”, means any molecule as defined above comprising an RNA moiety, such as siRNA (small interfering RNA), shRNA (short hairpin RNA), miRNA (microRNA), said molecules optionally comprising LNA nucleotides and/or being complexed with trans-membrane carriers such as cholesterol or cholesterol-derived molecules.

According to the present invention the general term “antibody” when used in the description includes an antibody, a humanised antibody, a fully human antibody, a Fab fragment, a F(ab′)₂ fragment, a single chain antibody.

Within the meaning of the present invention, the term “integrin(s)” is used in its standard meaning and thus, refers to protein receptors, expressed on the surface of cells that are responsible of outside-in and inside-out cell signaling. The term “integrin alpha 7” refers to the alpha 7 subunit of the cell surface expressed molecule, when reference is made to the protein ITGA7 the term does not exclude that the alpha subunit might be in an heterodimeric form such as ITGA7/ITGB1 on the cell surface. The ITGA7 can be any human isoform of the protein coded by the HGNC:6143 human integrin alpha 7 gene (Human Gene Nomenclature Committee) (e.g. transcript variant 1, transcript variant 2), by way of example can be it can be the ITGA7 coded by the NCBI mRNA reference sequence NM_{—}00144996.1, or by the NCBI mRNA reference sequence NM_{—}002206.2 or any other functional variant (isoform) of the ITGA7.

### DETAILED DESCRIPTION OF THE DRAWINGS

FIG. 1: In house developed Anti-integrin alpha 7 antibody binds preferentially on primary BTSC when compared to long term established brain tumour cell lines

1a) the binding pattern of the antibody produced by clone α-ITGA7-1 on primary brain tumour stem cell lines is shown and, in 1b), the binding pattern on differentiated, brain tumour cell lines. The binding was determined by flow cytometry and representative results are shown. T98G, U87MG, LN215 are lines obtained from GBM, Kelly, SHEP and SH-SY5Y from neuroblastoma.

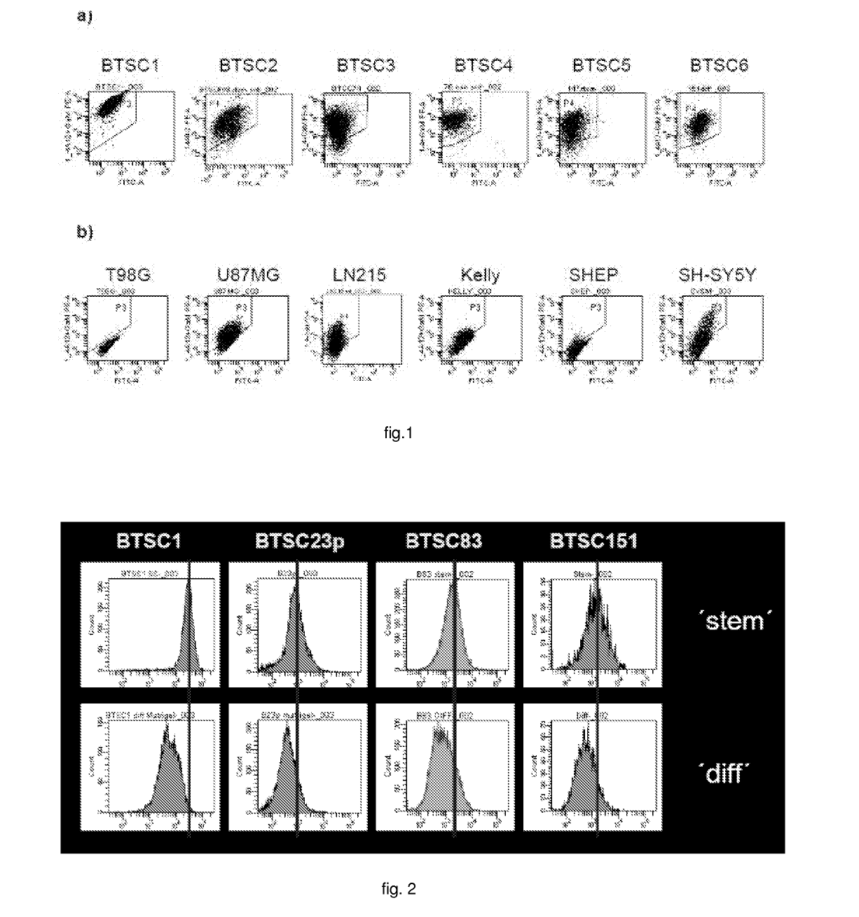

FIG. 2: Immunoreactivity of α-ITGA7 antibody on differentiated BTSCs is reduced compared to their stem-like counterpart

The binding pattern on 4 independent BTSC clones under stem-like and differentiation culture conditions is shown. The binding was determined by flow cytometry and representative results are shown. The cells were differentiated in the presence of 5% serum for 10-12 days.

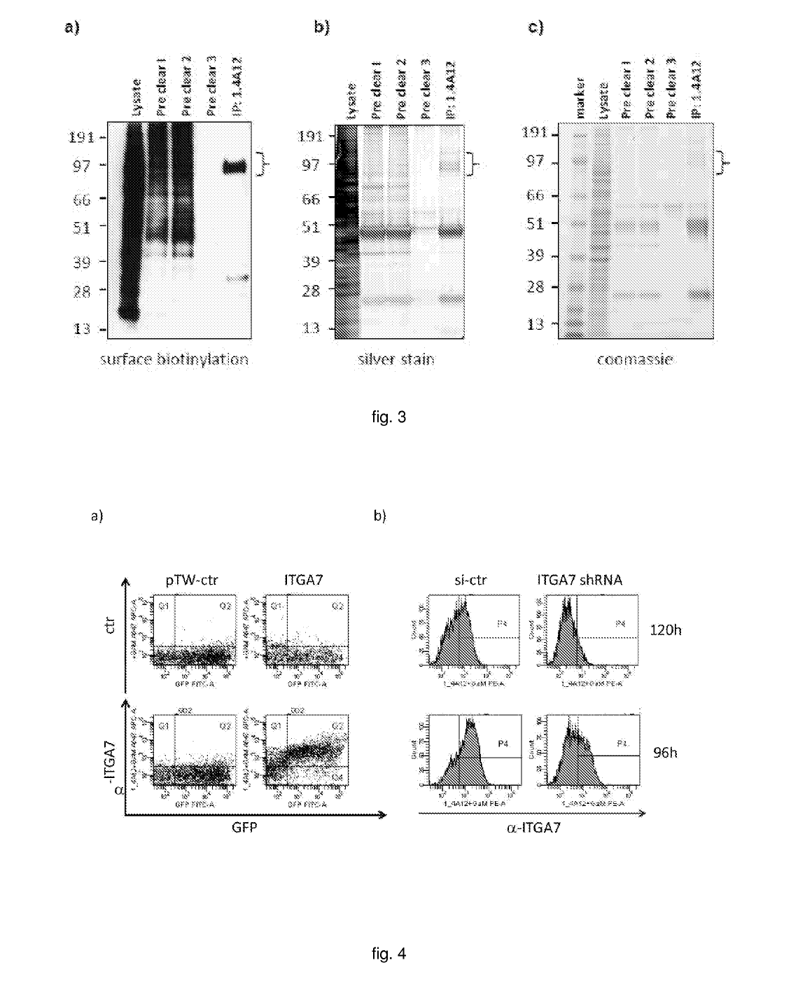

FIG. 3: Biochemical isolation of the α-ITGA7 antigen

3a) 2×10⁶ BTSC1 cells were first surface biotinylated using Sulfo-NHS-LC-Biotin (Pierce) and then subjected to immunoprecipitation using either IgG2a control antibody (preclears) or the α-ITGA7 antibody coupled to protein G beads. Shown is a western blot analysis, probed with streptavidin-HRP in order to detect the biotinylated antigen. 3b) The IP was repeated with 5×10⁶ BTSC1 cells and the isolated proteins were visualized by SDS PAGE and silver staining. 3c) Coomassie staining of the SDS-PAGE with the preparative immunoprecipitation, used for mass spectrometric analysis. The brackets show the bands with the potential α-ITGA7 antigen(s).

FIG. 4: α-ITGA7 antibody recognizes integrin alpha7

4a) HEK293T cells were transiently transfected with an expression plasmid for GFP (pTW-ctr) or ITGA7. 48 hrs post transfection the cells were harvested and a flowcytometric analysis using either a control (ctr) or the α-ITGA7 antibody was performed. 4b) BTSC1 cells which display a strong positivity for α-ITGA7 binding were transduced with either control (ctr) or ITGA7 shRNA. 120 hours post transduction a flowcytometric analysis using α-ITGA7 antibody was performed. The results shown are representative for at least 3 biological replicates.

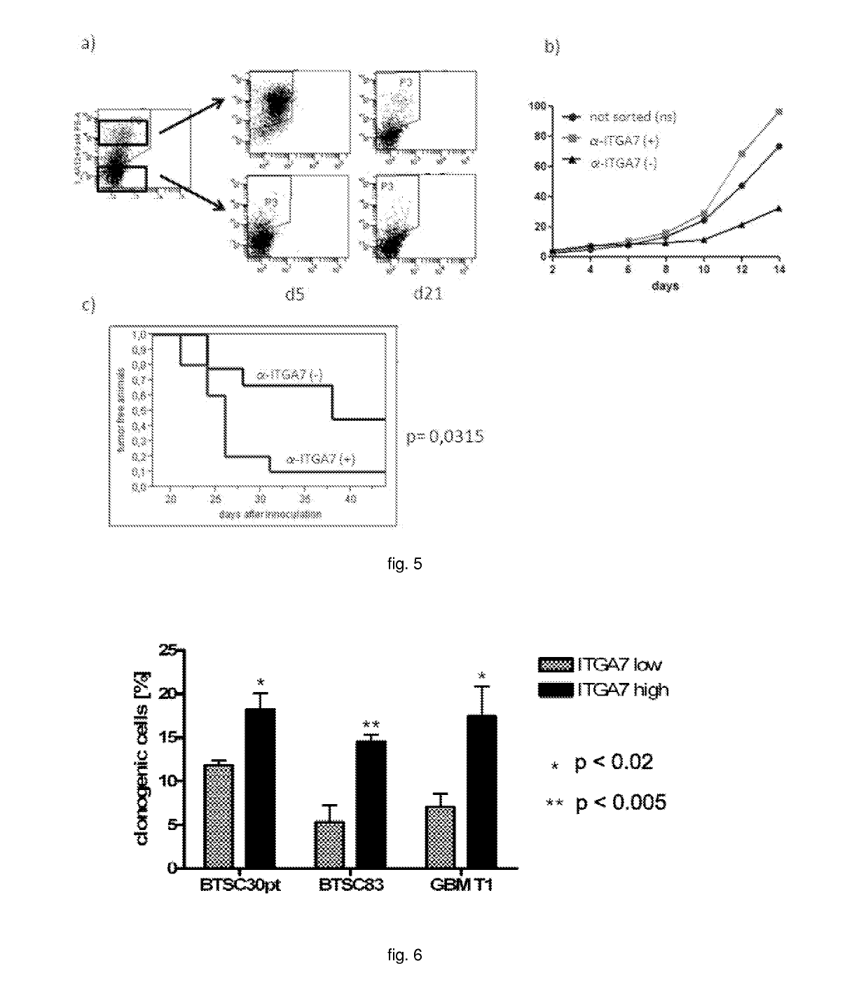

FIG. 5: The ITGA7 positive population of SH-SY5Y cells is enriched for tumourigenic cells

5a) SH-SY5Y cells were FACS sorted for ITGA7 positive and negative subpopulations. The expression of ITGA7 was controlled 5 and 21 days after the initial sorting by flowcytometry using the anti ITGA7 antibody. 5b) Not sorted (ns), ITGA7 positive (+) and negative (−) SH-SY5Y were seeded at a density of 1000 cells/well in a 96 well plate and the cell proliferation was determined every two days using an ATP-based assay (cell titer GLO). Plotted is the fold increase of ATP content compared to day 0. 5c) 5×104 SH-SY5Y positive or negative for ITGA7 expression were subcutaneously injected in CD1nude mice. The tumour development was monitored over a period of 50 days. The Kaplan-Meier plot shows the relative proportion of tumour free mice.

FIG. 6: The ITGA7 positive population of primary BTSC cells is enriched for clonogenic cells

Three glioblastoma stem cell lines (BTSC30pt, BTSC83 and GBM T1) were FACS sorted for ITGA7 positive and negative subpopulations. ITGA7 highly positive (high) and ITGA7 dim (low) cells were seeded at densities of 1 and 3 cells/well in a 96 well plate. The clonogenic capacity was determined 4 weeks after the plating. The wells containing clones were counted and the percentage of clonogenic cells was calculated. The average and SEM of 3-4 independent laydown experiments performed by flowcytometry was plotted.

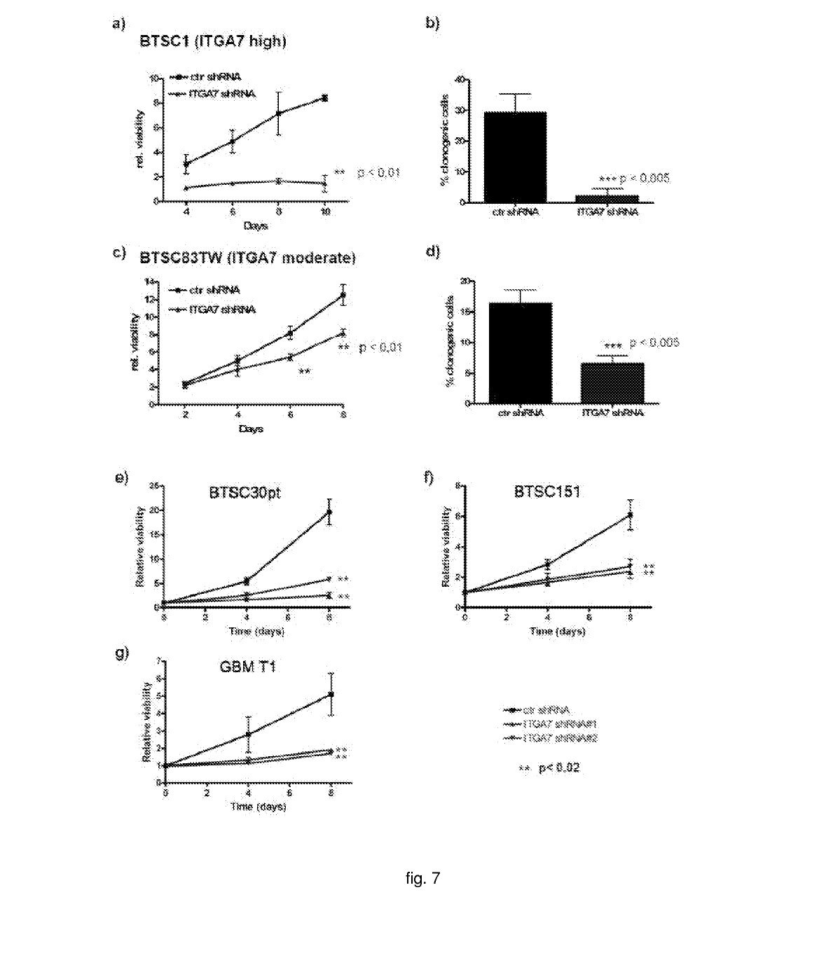

FIG. 7: Inhibition of ITGA7 impairs the growth and clonogenicity of BTSC1 in vitro

7a) BTSC1 cells were infected with lentiviral particles either encoding for a control small hairpin RNA (ctr-shRNA) or for shRNA targeting ITGA7 expression. Cells were seeded at densities of 500 cells/well in a 96 well plate and the cell proliferation was determined at the indicated time points using an ATP-based assay (cell titer GLO). The fold increase of ATP levels compared to day 0 was plotted. Average and SEM of 3 independent experiments are shown (Student t test: **p<0.01, ***p<0.005). 7b) Cells treated like in 7a) were seeded at a density of 1 cell/well in 96 well plates. 21 days after plating the wells were controlled for colonies and shown are average and SD of colonies detected in 4 independent experiments. 7c) and 7d): same experiment as described in 7a) and 7b) using a second primary brain tumour stem like cell line (BTSC83). 7e-g): Growth curves as in 8a) performed with 3 additional primary glioblastoma stem like cells. ITGA7 was knocked down using two independent shRNA constructs. Average and SEM of 3 independent experiments are shown. (Student t test: **p<0.02)

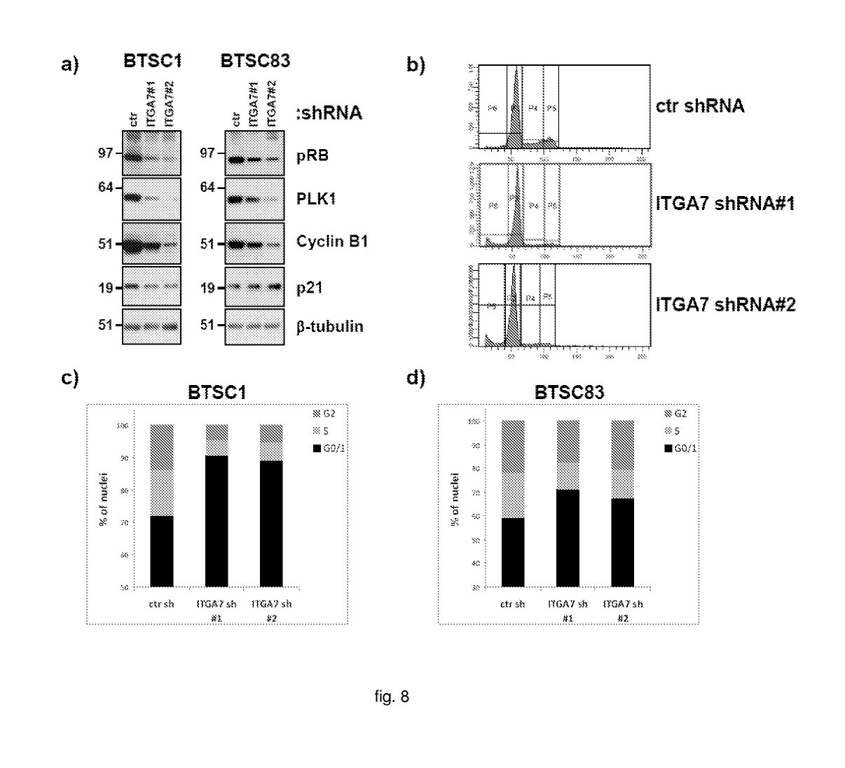

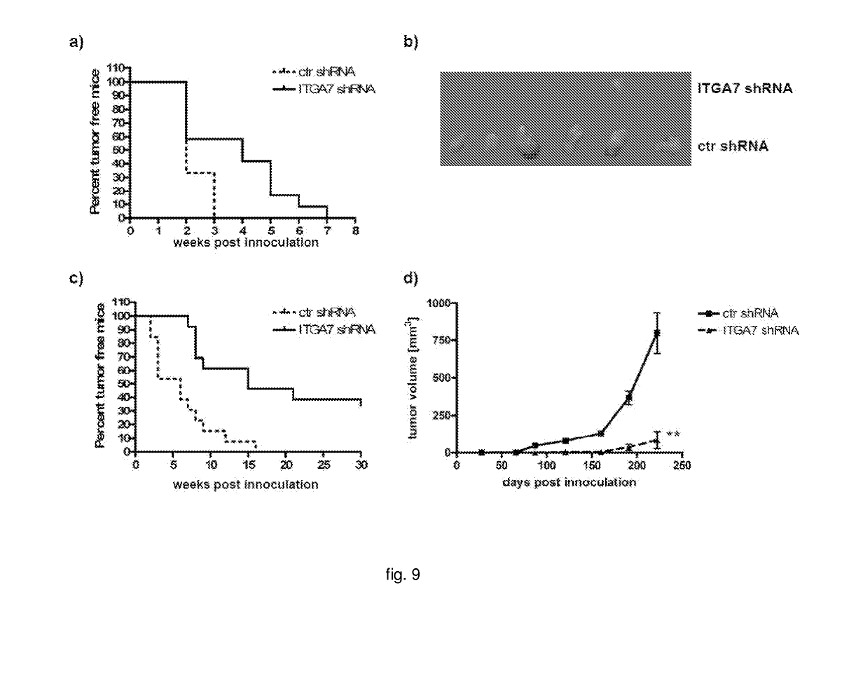

FIG. 8: ITGA7 knockdown leads to cell cycle arrest in glioblastoma stem like cells

8a) Shows a western blot analysis of BTSC1 and BTSC83 cells either transfected with control shRNA or two independent ITGA7 targeting shRNAs. The membranes were probed with the antibodies recognizing cell cycle dependent proteins indicated. 8b) BTSC1 cells were infected with lentiviral expression constructs for ctr shRNA or two independent shRNAs targeting ITGA7. 9-11 days after transduction cell cycle analysis was performed. Representative histograms obtained by flowcytometry are shown. 8c) Represents the analysis of the cell cycle analysis described in 8b). 8d) Shows the block in cell cycle progression upon ITGA7 knockdown in a second glioblastoma stem like cell line (BTSC83).

FIG. 9: ITGA7 knockdown impairs tumour growth in xenograft model

9a) The formation of palpable nodules upon injection of 100000 BTSC1 either transduced with control shRNA (ctr shRNA) or shRNA targeting ITGA7 is shown. 9b) BTSC1 tumours were explanted 30 weeks post inoculation. Shown is the harvest of one out of two cages. 9c) Formation of palpable nodules upon injection of 100000 BTSC83 either transduced with control shRNA (ctr shRNA) or shRNA targeting ITGA7. 9d) growth curve of engrafted BTSC83 transduced with ctr or ITGA7 shRNA. ** indicate a p value of <0.02 (two tailed student t test).

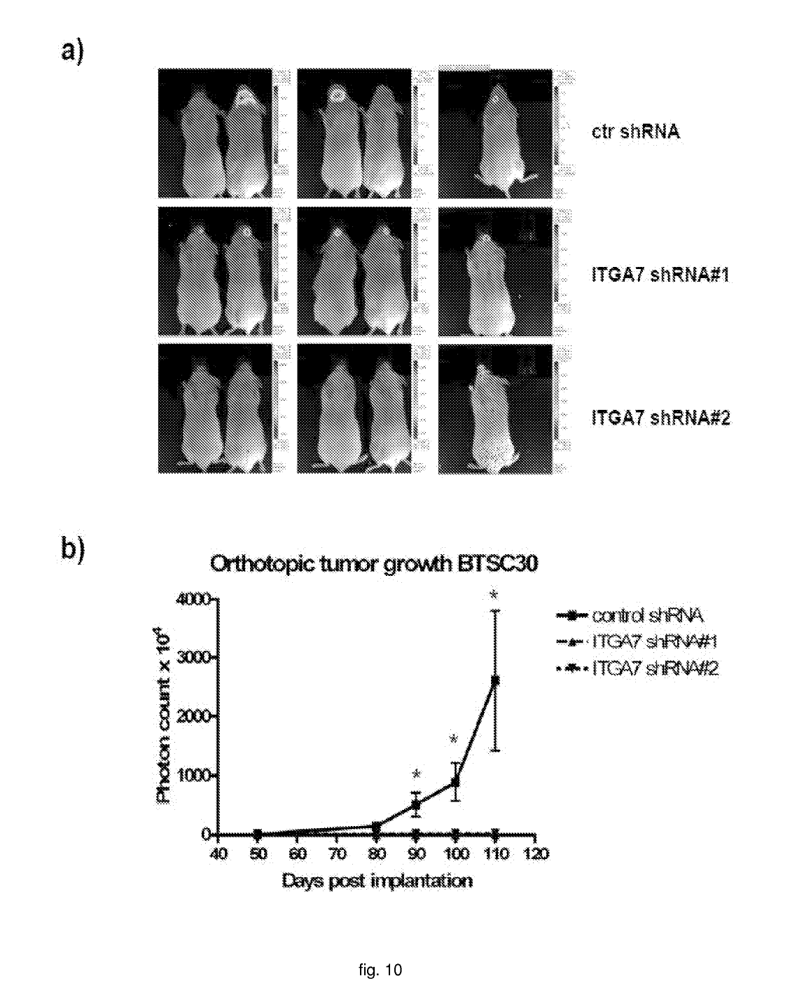

FIG. 10: ITGA7 knockdown impairs tumour growth in orthotopic xenograft model

Mice were intracranially injected with 30000 BTSC30pt cells carrying the luciferase-gene. These cells were additionally transduced with non targeting shRNA constructs (ctr) or two independent shRNA constructs inhibiting ITGA7 expression (ITGA7 shRNA). 10a) shows the luciferase activity detected with a Xenogen IVIS 100 small animal in vivo imaging system. 10b) shows the statistic analysis of orthotopic tumour growth with 4 and 5 mice/group as measured by luciferase activity; * indicate a p value of <0.05 (two tailed student t test).

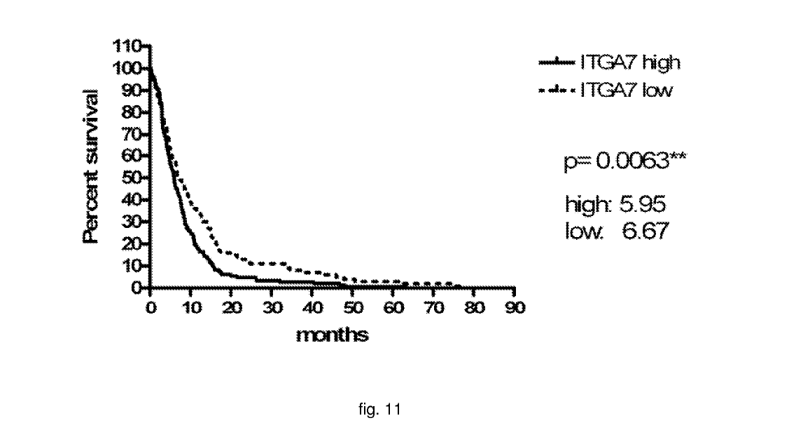

FIG. 11: Progression free survival of GBM patients with high ITGA7 is shorter compared to patients with low ITGA7 levels

Kaplan-Meier plot was generated from open source data from TOGA, which was re-analyzed in order to correlate ITGA7 expression and progression free survival of the patient. To define ITGA7 high expressing GBMs a threshold of a 1.7 fold enrichment of ITGA7 signal compared to the healthy tissue (peripheral blood lymphocytes) was set.

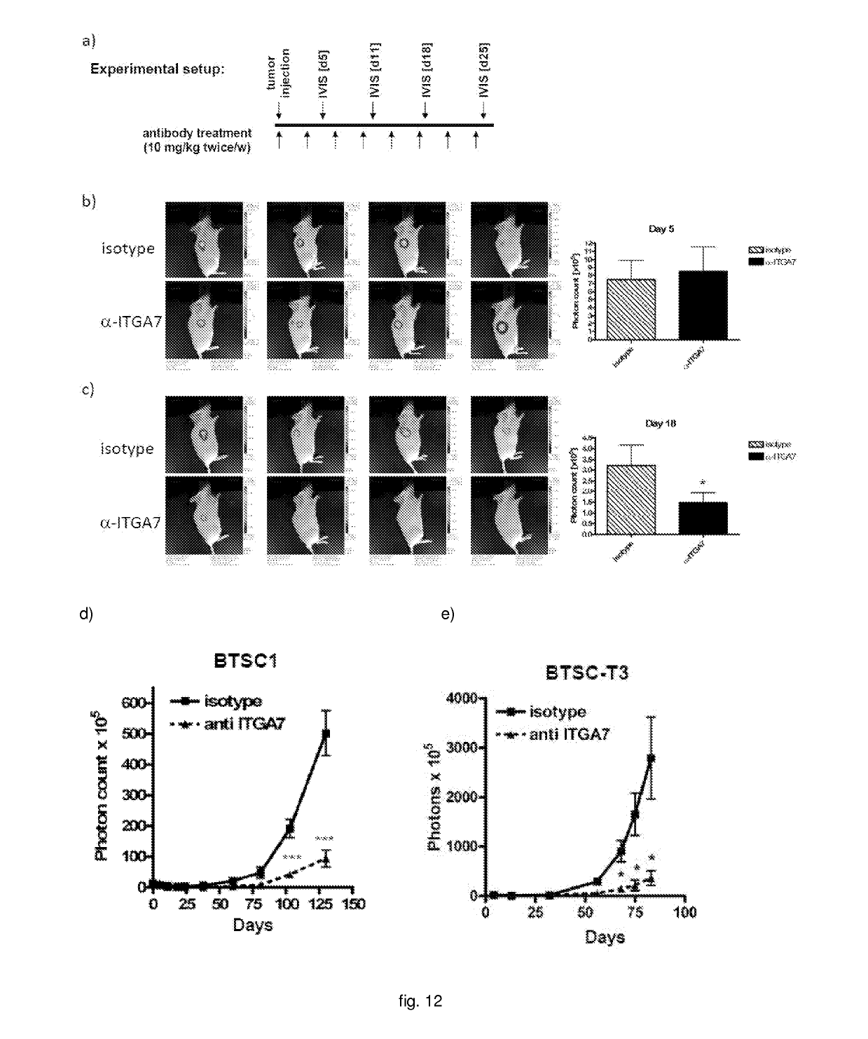

FIG. 12: Anti-ITGA7 antibody inhibits tumour engraftment in vivo

FIG. 12a) shows the experimental setup for the therapeutic treatment experiment with anti ITGA7 antibody. NSG mice were injected with 1×10⁶ luciferase expressing BTSC1 cells and 10 mg/kg anti integrin alpha 7 antibody or isotype control (6 mice/group). The antibody treatment was repeated two times/weekly. At the indicated time points tumour engraftment was determined by detection of luciferase activity using an IVIS Xenogen 100 small animal in vivo imaging system. In 12b) representative images and the statistical analysis of tumour engraftment 5 days post injection are shown. In 12c) representative images and the statistical analysis of the measured photons at day 18 clearly indicate a significantly reduced engraftment and/or growth of the tumours in the animals treated with the anti ITGA7 antibody. * indicates a p value of <0.05 (two tailed student t test).

FIGS. 12d) and e) show the experimental results of subcutaneous injection of NSG with 1×10⁶ luciferase expressing BTSC cells. FIG. 12d) shows the results with BTSC1 cells and FIG. 12e) shows the results with BTSC-T3 cells. The mice were simultaneously treated with systemic administration (i.p.) of 10 mg/kg anti integrin alpha 7 antibody (1.4A12) or isotype control (6 mice/group). The antibody treatment was repeated two times/weekly for a total of 4 weeks. At the indicated time points tumour engraftment was determined by detection of luciferase activity using an IVIS Xenogen 100 small animal in vivo imaging system.

*** and * indicate a p value of <0.05 and <0.001, respectively (two tailed student t test).

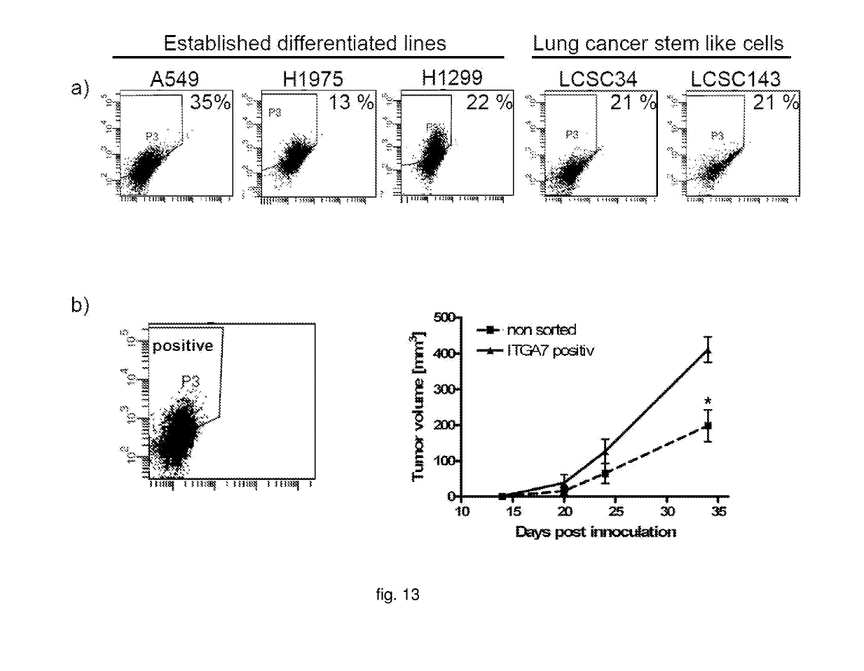

FIG. 13: Integrin alpha7 is also expressed by lung tumour cells and is a marker for a highly tumourigenic cell population

13a) Shows the ITGA7 surface expression on differentiated lung tumour cell lines and primary lung tumour stem like cell lines. 13b), H1299 cells were FACS sorted for the ITGA7 positive fraction, after the sorting cells were recovered for 1 hour on ice and 50.000 cells were subcutaneous injected into the flanks NSG mice. Tumour growth was determined at the time points indicated and 34 days post implantation a significant difference in cell growth was detected. (n=4 for ITGA7 positive cells and n=8 for bulk population. * indicates a p value of <0.05 (two tailed student t test).

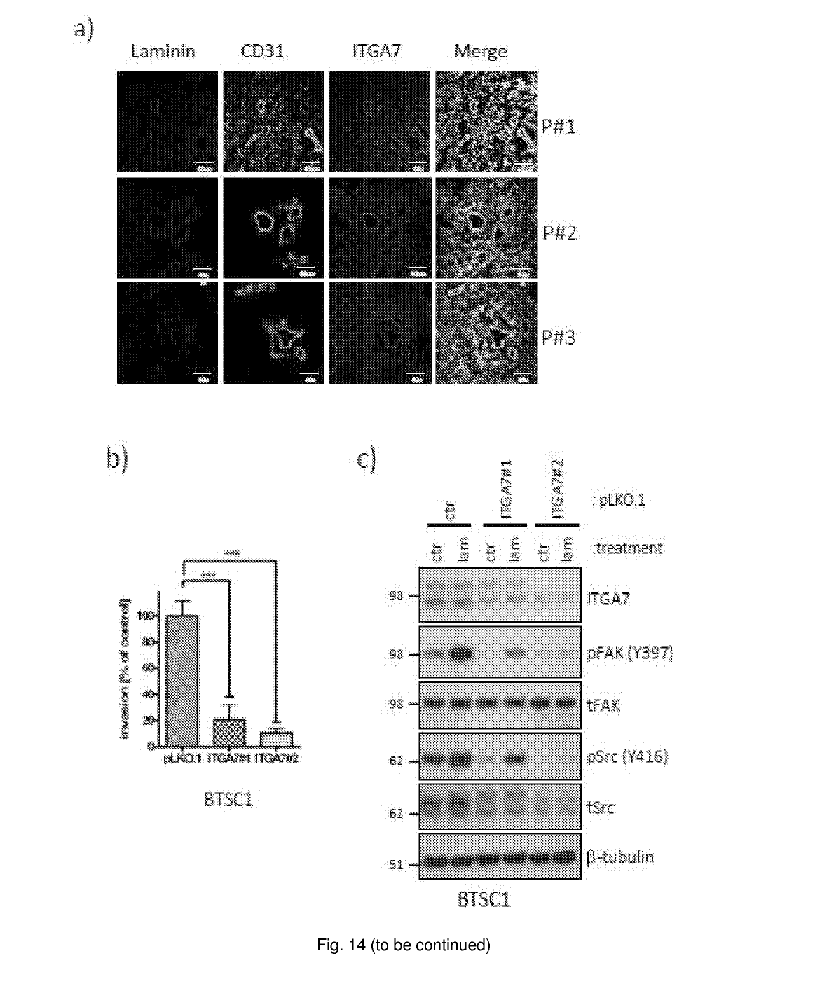

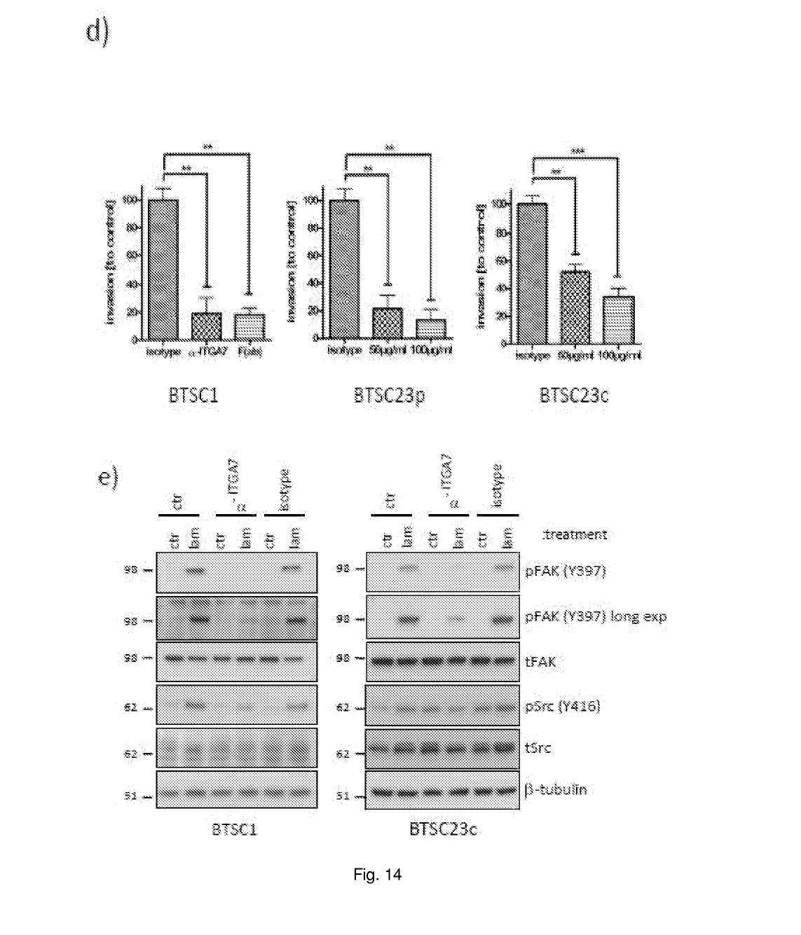

FIG. 14: ITGA7 is co-expressed with laminin in GBM and mediates laminin induced signaling and invasion in vitro.
a) Immunofluorescence picture of frozen tumour specimens from three different primary GBM stained with the antibodies indicated.
b) BTSC1 cells were treated with lentiviral particles containing control and ITGA7 targeting shRNAs (SEQ IDs 1 and 2). 10 Days after transduction 20000 cells/well were seeded and an invasion assays was performed in transwell chambers (BD fluoroblok) coated with laminin (10 μg/ml). After 48 hrs the cells were stained with Calcein AM. The photographic pictures of the migrated cells were then analyzed with ImageJ. Shown are average and SEM of 3 independent experiments done in triplicate. *** indicate a p value of <0.001 (ANOVA).
c) BTSC1 cells treated with ctr or ITGA7 shRNA containing lentiviral particles were incubated on laminin coated plates for 30 min. Afterwards the cells were lysed and western blot analysis was performed. The membrane was stripped after every detection with the antibodies indicated.
d) The invasion of BTSC1, BTSC23c and BTSC23p in presence or absence of blocking ITGA7 antibody (anti ITGA7) or the corresponding F(ab) fragment (F(ab)) was measured in transwell chambers (BD fluoroblok) coated with laminin (10 μg/ml). After 48 hrs the cells were stained with Calcein AM. The photographic pictures of the migrated cells were then analyzed with ImageJ as described in M+M. Shown are average and SEM of 3 independent experiments done in triplicate. ** and *** indicate a p value of <0.01 and <0.001, respectively (ANOVA).
e) BTSC1 or BTSC23c cells were preincubated with CSC medium either containing 50 μg/ml anti ITGA7 (1.4A12), non-binding isotype control antibody or PBS for 15 min at 37° C. Then the cells were seeded on laminin coated plates for 30 min. Afterwards the cells were lysed and western blot analysis was performed. The membrane was stripped after every detection with the antibodies indicated.

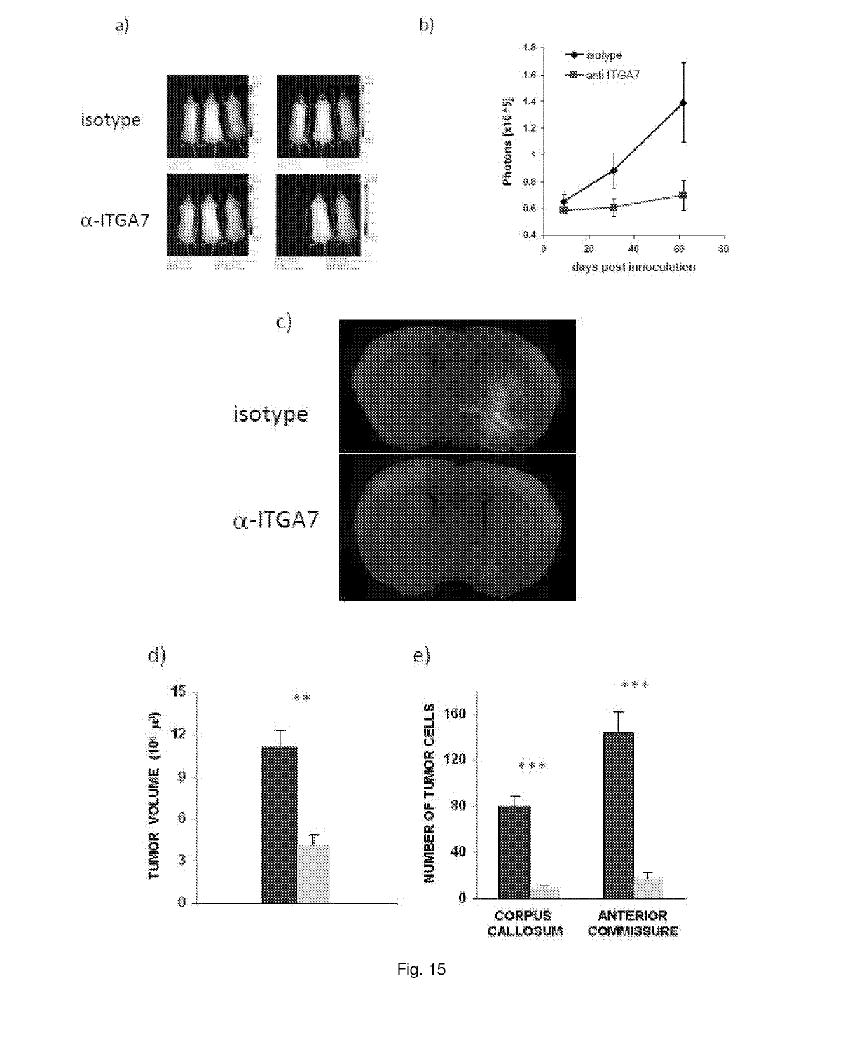

FIG. 15: Antibody mediated ITGA7 blockade significantly impairs GBM tumour engraftment and invasion in an intracranial model.
a) Luciferase activity was detected in mice 40 days after intracranial transplantation of 5×10⁴ BTSC1-LUC cells using a Xenogen IVIS 100 small animal in vivo imaging system. The cells were coinjected with either 3 μg anti ITGA7 or isotype control antibody. Afterwards mice were either treated with 10 mg/kg anti ITGA7 antibody (1.4A12) or vehicle (isotype) twice weekly for 28 days.
b) Analysis of tumour growth over time of the mice shown in a). Shown is the average photon count and SEM of 6 or 5 mice/group as measured by Xenogen IVIS 100 small animal in vivo imaging system.
c) Representative picture of a mouse brain slice showing the location of the GFP positive tumour cells 8 weeks after intracranial injection of 5×10⁴ BTSC1-LUC. The systemic antibody treatment was performed for 4 weeks twice weekly.
d) Statistical analysis of the tumour volumes shown in c). The dark grey bars represent the control, while the light grey bars represent the anti ITGA7 treated group. Shown is average and S.E.M of 4 mouse brains (** p<0.005; student t test)
e) Number of tumour cells that infiltrated the corpus callosum and the anterior commissure. The dark grey bars represent the control, while the light gray bars represent the anti ITGA7 treated group. Shown is average and S.E.M of 4 mouse brains (*** p<0.001, student t test).

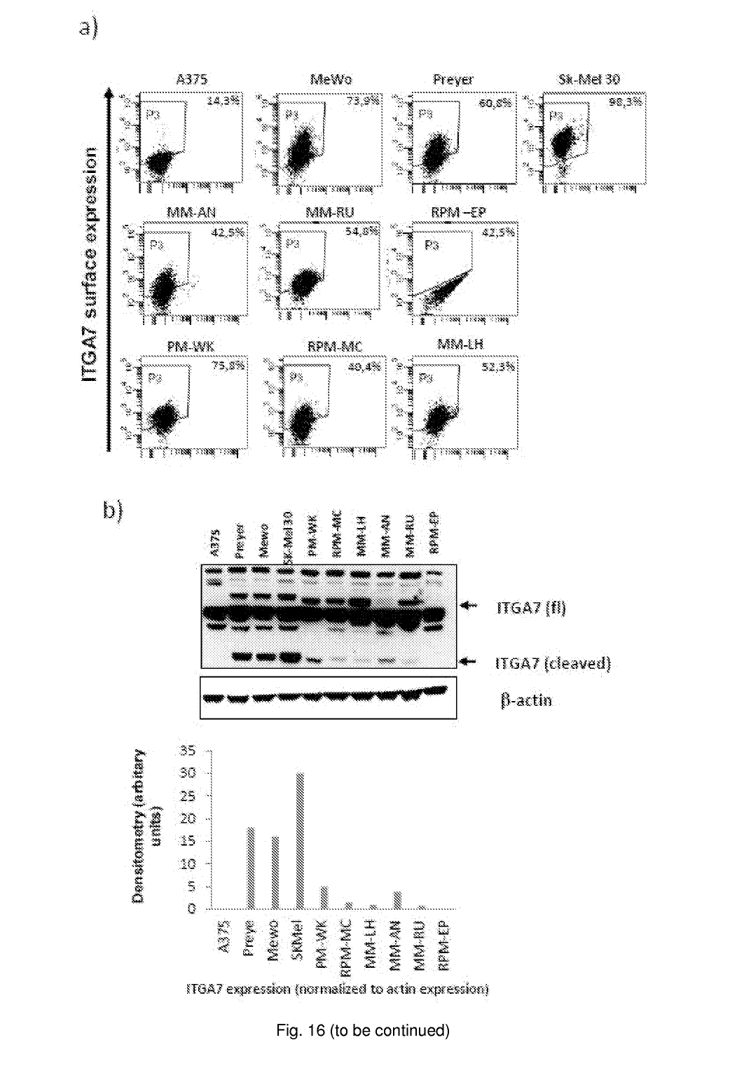

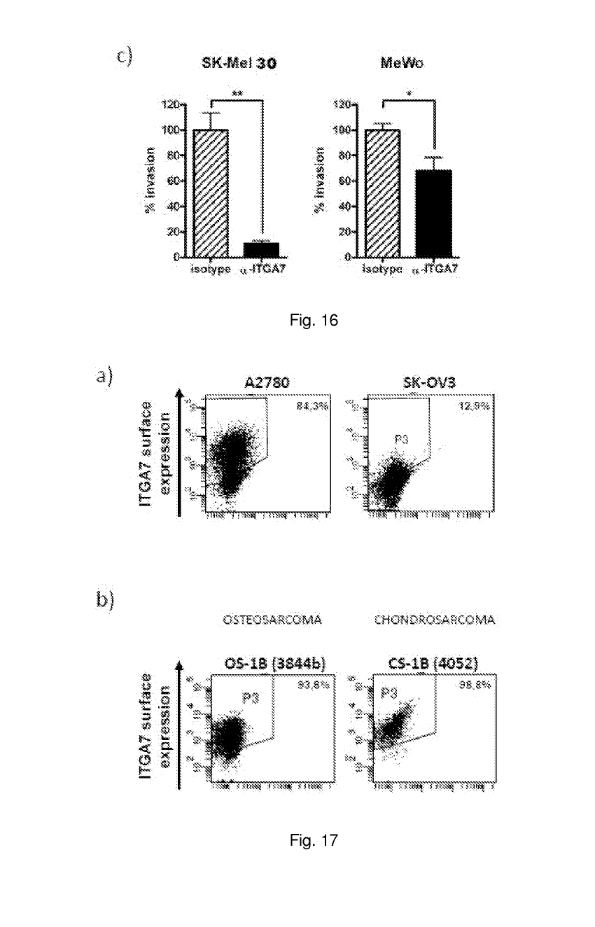

FIG. 16: Melanoma cell lines express ITGA7 and antibody mediated blocking of ITGA7 significantly inhibits cell invasion.
a) Binding pattern for 1.4A12 anti ITGA7 antibody on 10 independent melanoma cell lines. The binding was determined by flow cytometry and representative results are shown. The fractions of ITGA7 positive cells are between 14.3 and 98.3% of the total cell population.
b) Western blot analysis of the ITGA7 expression using a polyclonal antibody. The arrows indicate the specific band for ITGA7. In the lower part the signal for ITGA7 is normalize to the signal for actin expression in the same samples
c) Laminin invasion assay for two melanoma cell lines (MeWo and SK-Mel 30). 4×10⁴ melanoma cells were seeded in presence of 50 μg/ml anti ITGA7 or control antibody (isotype) in a Fluoroblok migration chamber which was coated with 20 μg/ml laminin in PBS. in the lower chamber 100 ng/ml EGF was used as chemoattractant. 72 hours after the seeding the cells were stained with 2 μM Calcein AM and the fluorescent cells were monitored under the microscope. The microscopic pictures were analyzed with imageJ and normalized to the isotype control. Shown is average and SEM of a representative experiment done in triplicates. (* p<0.05; ** p<0.005, student t test).

FIG. 17: Immunoreactivity for ITGA7 on a panel of melanoma cell lines and primary chrondro- and osteosarcoma stem like cells
a) Binding pattern for 1.4A12 anti ITGA7 antibody on two independent ovarian cancer cell lines [A2780 and SK-OV3, respectively]. The binding was determined by flow cytometry and representative results are shown. The fractions of ITGA7 positive cells are more than 84.3% and 12.9% respectively.
b) Binding pattern for 1.4A12 anti ITGA7 antibody on primary osteosarcoma and chondrosarcoma stem cell lines [OS-1B and CS-B, respectively]. The binding was determined by flow cytometry and representative results are shown. The fractions of ITGA7 positive cells in these primary sarcoma cells are more than 90% in both cases.

### DETAILED DESCRIPTION OF THE INVENTION

Object of the present invention is hence an inhibitor of the integrin alpha 7 activity for use in the treatment and/or prevention of tumours.

The inventors have demonstrated in the experimental section below that integrin alpha 7 is expressed on the cell surface of different highly aggressive tumour cells obtained from different tumour samples such as glioblastoma multiforme, lung cancer and other cancers. The inventors did in fact observe ITGA7 expression in 10 different cell lines of glioblastoma including primary lines, 5 different cell lines of lung cancer including primary lines, 1 line of neuroblastoma in which the ITGA7 high expressing fraction of cells is very aggressive compared to the cells bulk, 10 lines of melanoma, 2 lines of ovarian cancer and 2 primary lines of sarcoma.

Inhibition of the integrin alpha 7 activity, either indirectly through the inhibition at the expression level, or directly, through inhibition of the protein via antibody binding, has proven to inhibit the tumour cell proliferation in vitro and the tumour growth in vivo in mice experimental models.

The data obtained by the inventors indicate that the inhibition of integrin alpha7 function negatively influences the proliferation of primary tumour stem cell like cells directly. In addition the phenotype of ITGA7 knockout mice indicate only minor effects on non-tumour tissue, such as the vascular system, as especially vascularization is heavily altered in mice knocked out for other integrin subunits. In contrast to DNA-damaging irradiation or conventional chemotherapy, the inhibition of integrin alpha7 primary leads to a block in cell cycle progression, affecting only actively cycling cells without the risk of inducing mutations in the normal tissue. This might reduce the risk for secondary tumours initiated by the anti-cancer treatment.

The inhibitors of the invention are hence useful in the treatment and/or in the prevention of tumours as they inhibit the tumour growth and strongly decrease the aggressiveness of the tumour cells. In fact, tumour cells expressing integrin alpha 7 on the cell surface were found to be highly aggressive as also shown in the comparative assay. The inhibitors of invention will hence have a therapeutic effect against tumour engrafting and growth.

According to the invention, the treatment of the tumour may result in a partial or total tumour regression, in a decrease of the growth rate of the tumour, in a decrease of the tumour aggressiveness. Furthermore, the inhibitor of the invention may prevent the development of tumour, e.g. in patients where a tumour was ablated or is under treatment and a substantial risk of metastatic growth exists, by decreasing the aggressiveness and the clonogenicity of the tumour cells.

According to the invention the tumour can be any tumour expressing ITGA7 on the surface of at least a population of tumour cells. It is in fact known that tumours arise from a series of mutations occurring in few or single founder cells. Transformed stem cells also known as Cancer stem cells (CSCs) are cancer cells (found within tumours or hematological cancers) that possess characteristics associated with normal stem cells, specifically the ability to give rise to all cell types found in a particular cancer sample. CSCs are therefore tumourigenic (tumour-forming), perhaps in contrast to other non-tumourigenic cancer cells. CSCs may generate tumours through the stem cell processes of self-renewal and differentiation into multiple cell types. Such cells are proposed to persist in tumours as a distinct population and cause relapse and metastasis by giving rise to new tumours. In an embodiment of the invention the at least a population of tumour cell can be a cancer stem cells or tumour stem cells population.

According to the invention, the tumour treated and/or prevented by the inhibitor of integrin alpha 7 activity can be a CNS tumour such a tumour selected from primary brain tumours or lower grade brain tumours, Head/Neck (Oral, Nasopharyngeal) tumours, digestive system tumours, respiratory system tumours, bone tumours, skin tumours, blood tumours, urogenital tumours, nervous system tumours, endocrine system tumours, sarcomas and gynaecological cancers.

According to the invention the tumour can be a carcinoma, a sarcoma, a blastoma, a papilloma an adenoma.

With reference to brain tumours according to the invention the brain tumour might be selected from astrocytic tumours such as glioblastoma multforme (giant cell glioblastoma, gliosarcoma), glioma, astrocytoma (protoplasmic, gemistocytic, fibrillary, mixed); oligodendroglial tumours such as oligodendroglioma, Anaplastic (malignant) oligodendroglioma; ependymal cell tumours, such as ependymoma (cellular, papillary, epithelial, clear cell, mixed), Anaplastic ependymoma, Myxopapillary ependymoma, Subependymoma; mixed gliomas such as mixed oligoastrocytoma, anaplastic (malignant) oligoastrocytoma, Others (e.g. ependymo-astrocytomas); Neuroepithelial tumours of uncertain origin such as polar spongioblastoma; astroblastoma; Gliomatosis cerebri; Tumours of the choroid plexus such as Choroid plexus papilloma; Neuronal and mixed neuronal-glial tumours such as gangliocytoma, dysplastic gangliocytoma of cerebellum (Lhermitte-Duclos), ganglioglioma, anaplastic (malignant) ganglioglioma, Desmoplastic infantile ganglioglioma, Desmoplastic infantile astrocytoma, central neurocytoma, dysembryoplastic neuroepithelial tumour, olfactory neuroblastoma (esthesioneuroblastoma); Primitive neuroectodermal tumours with multipotent differentiation, medulloblastoma (medullomyoblastoma, melanocytic medulloblastoma, desmoplastic medulloblastoma), cerebral primitive neuroectodermal tumour, Neuroblastoma (ganglioneuroblastoma), Retinoblastoma, Ependymoblastoma

Non small cell lung cancer (NSCLC) including lung adenocarcinoma, lung squamous cell carcinoma and lung large cell carcinoma., colon carcinoma, breast carcinoma, all types of melanoma, all types of ovarian cancer and sarcomas such as chondrosarcoma or osteosarcoma, Ewing's sarcoma and soft tissue sarcomas such as leiomyosarcoma and rhabdomyosarcoma.

In one embodiment according to the invention, the tumour can be of neuroectodermal origin (e.g. glioblastoma multiforme or melanoma), a mesenchymal tumour (sarcoma), an epithelial tumour (e.g. lung tumours) or a tumour showing a epithelial-mesenchymal transition, hence acquiring, eventually partially, a mesenchymal phenotype (e.g. expression of one or more mesenchymal marker and/or loss of epithelial markers etc.).

According to the invention, the inhibitor can be any molecule or group of molecules impairing the activity or the functionality of human integrin alpha 7.

In other words, the inhibitor may act indirectly, e.g. at an expression level, or directly, e.g. at the protein level. An inhibition at the expression level can be carried out at the transcriptional or at the translational e.g. by epigenetic inhibition or by RNA interference techniques. RNAi application is well known to the skilled person and has reached up to date clinical trials e.g. in the treatment of macular degeneration and respiratory syncytial virus, RNAi has also been shown to be effective in the reversal of induced liver failure in mouse models and is at present proposed for several antiviral therapies. RNA interference is also often seen as a promising way to treat cancer by silencing genes differentially upregulated in tumour cells or genes involved in cell division. The skilled person, starting for the known mRNAs coding for ITGA7 (see e.g. glossary above), can easily design a large number of molecules to use as RNA-based inhibitors.

The data provided in the present application (cfr. in vitro and in vivo results examples 13 and 14) show that RNA inhibition with RNA-based inhibitors targeting the mRNA of human integrin alpha 7 drastically reduce the tumour cells proliferation in vitro and that the in vivo tumour growth in mice in which RNAi of ITGA7 expression was carried out was either inexistent or strongly delayed. Hence, according to an embodiment of the invention the inhibitor can be an RNA based inhibitor exerting its activity by RNA interference.

In an embodiment of the invention the RNA-based inhibitor can be selected from shRNA, siRNA, miRNA. The RNAi technique is well known in the art and reported as feasible for therapeutic use in mammals.

In another embodiment the RNA-based inhibitor of the invention can be a shRNA of SEQ ID NO 1 or of SEQ ID NO 2.

The efficacy of two methods of systemic siRNA delivery and the effects of siRNA modifications using locked nucleic acids (LNA) in a xenograft cancer model has been compared in the art. It has also been shown that LNA can be incorporated into the sense strand of siRNA while the efficacy is retained. Modification of siRNA targeting green fluorescent protein with LNA results in a significant increase in serum stability and thus may be beneficial for clinical applications. It has been shown, by way of example, that minimal 3′ end LNA modifications of siRNA are effective in stabilization of siRNA. Multiple LNA modifications in the accompanying strand further increase the stability but negate the efficacy in vitro and in vivo. In vivo, LNA-modified siRNA reduced off-target gene regulation compared with nonmodified siRNA. End-modified siRNA targeting green fluorescent protein provides a good trade-off between stability and efficacy in vivo using the two methods of systemic delivery in the nude mouse model. Hence, according to one embodiment of the invention, the RNA-based inhibitor may comprise LNA nucleotides. LNA modified oligomers are synthesized chemically and are commercially available. The locked ribose conformation enhances base stacking and backbone pre-organization.

RNA-based inhibitors have also been conjugated in the art to various molecules in order to improve the delivery of the same in the cells. By way of example, conjugation approaches have shown promise in facilitating cellular entry of siRNAs. This may involve conjugating siRNAs to small peptides that can penetrate cellular membranes, or to simple small molecules such as a cholesterol moiety. Cholesterol-conjugation of siRNAs was shown to provide a functional in vivo delivery of an siRNA following systemic administration in mice. Cholesterol-conjugation has the added benefit of functioning as a carrier for the siRNA in the blood thereby improving its pharmacokinetic properties. This is of particular importance for the systemic delivery of RNAi therapeutics.

Hence, in one further embodiment, the RNA-based inhibitor of the invention may be cholesterol-siRNA conjugate.

In yet another embodiment the RNA-based inhibitor of the invention may be inserted in an adenoviral, lentiviral or retroviral vector for delivery, according to the teachings available in the art. The skilled person would know, without use of inventive skill how to insert the RNA-based inhibitor of the invention on one of the well-known cited vectors above.

In a different embodiment, the inhibitor of the invention may exert its inhibitory effect by acting on the ITGA7 protein or on the ITGA7/ITGB1 heterodimer.

By way of example the inhibitor of the invention can be an antibody binding the extracellular domain of the protein and blocking protein function by interfering with integrin activation such as an anti-ITGA7 antibody or it can be a small inhibiting molecule still interfering with the integrin activation or functionality. In one embodiment, the inhibitor may disrupt the interaction of the ITGA7 and ITGB1 subunits. In that case, due to the fact that inhibition of ITGB1 has been reported as causing severe side effects on mice, the inhibitor of the invention will act on the interaction ITGA7-ITGB1 by acting on the ITGA7 unit and without disrupting the ITGB1 subunit availability for forming functional dimers with other alpha integrins.

In a further embodiment the inhibitor of the invention will block the activation of the focal adhesion kinase (FAK) by the ITGA7/ITGB1 dimer.

In a further embodiment the inhibitor will interfere interfering with the binding to the natural ligand laminin, an example of such inhibitor can be, as already stated above, an anti-ITGA7 antibody or peptides described in the art such as the peptides derived from laminin B1 chain as described by Nomitzu et al in Cancer Research, vol 53, pp. 3459-3461 (see, e.g. title).

For the direct inhibiting activity as herein defined, the inhibitor may be an antibody, a Fab, a F(ab′)₂ fragment, a single chain antibody, a partially or fully humanized, or recombinant human antibody.

The feasibility of the generation of blocking antibodies is documented by the high number of publications describing antibodies blocking the function of other integrin subunits. The generation of blocking integrin alpha7 antibodies can be carried out by standard techniques. By way of example murine cells (L-cells) infected with lentiviral ITGA7 expression constructs can be used for the immunization of syngenic mice (C3H/An). This provokes immunoreactions directed against the human ITGA7 antigen and ensures in parallel the native expression of ITGA7. As for the generation of the anti ITGA7 antibody, hybridomas can be generated and subcloned one day after fusion a quick workflow can be guaranteed. The identification of functional antibodies can be performed, by way of example, in 3 distinct steps:

(I) an initial screening procedure by ELISA method, using the transgenic ITGA7 expressing L-cells as bait. Positive clones will be re-tested on native L-cells by the same method.

(II) The positive supernatants will be re-screened by FACS, using ITGA7 positive and negative human cell lines.

(III) Finally the functionality of the antibody will be determined by two different tumour cell based cell based assays:

The antibody mediated inhibition of the binding of the cells to laminin can be determined by an in vitro attachment assay, the direct influence of hybridoma supernatants on cell viability can be determined by using a cell titer GLO viability assay.

The screening methods described here are straight forward and easy to perform in high (2000-3000 tests) and medium (100-200 tests) throughput scale for ELISA and cell based assays, respectively.

Methods for Fab, a F(ab′)₂ fragment, a single chain antibody, a partially or fully humanized, or a recombinant human antibody generation is also well known in the art.

According to the invention pharmaceutical composition comprising the inhibitor of the invention and at least one pharmaceutically acceptable carrier are provided.

The compositions can be in form of injectable compositions or in the form of compositions for oral or topic administration.

These compositions may obviously comprise one or more pharmaceutically acceptable vehicles, diluents and/or excipients. The compositions can be in any form deemed appropriate by the skilled person, such as solid, semi-solid, liquid, granular, and all suitable forms known to the skilled person.

The liquid forms may be appropriate forms for oral or systemic administration.

Pharmaceutical compositions suitable for oral administration can be capsules, tablets, pills, powders, granules, solutions or suspensions in aqueous or non-aqueous liquids, foam or beaten edible, liquid oil in water emulsions or liquid water in oil emulsions.

For example, for oral administration in capsule, gelatine or tablet form, the compounds mentioned above may be combined with a non-toxic pharmaceutically acceptable inert carrier such as ethanol, glycerol, water and similar. There may also be present flavourings, preservative, colouring and dispersant agents.

A pharmaceutical composition suitable for oral administration in capsule form can be prepared using encapsulation procedures. For example, pellets containing the active ingredient may be prepared using a suitable pharmaceutically acceptable vehicle and then be placed in a hard gelatine capsule. Alternatively, a dispersion or suspension may be prepared using any suitable pharmaceutically acceptable vehicle, such as an aqueous rubber or an oil and the dispersion or suspension can then be placed in a soft gelatine capsule.

The composition may be in unitary dose form such as a tablet or capsule for oral administration, for example, for oral administration to a human being. Where appropriate, dosage unitary formulations for oral administration may be micro encapsulated. The formulation can also be prepared to prolong or maintain the release by way of example by coating or by embedding particulate material into polymers, waxes or the like.

Liquids for oral use as solutions, syrups and elixirs can be prepared in the form of dosage units so that a given quantity contains a predetermined quantity of the compounds mentioned above. Generally a liquid formulation consists of a suspension or solution of the compounds mentioned above in one or more pharmaceutically liquid suitable vehicles, such as an aqueous solvent such as water, ethanol or glycerine, or a non-aqueous solvent, such as polyethylene glycol or oil. The formulation may also contain a suspending agent, preservative, flavouring and/or dye.

Compositions suitable for parenteral administration may include sterile aqueous or non-aqueous solution for injection which may contain antioxidants, buffers, bacteriostatic and solutes which render the solution isotonic with the blood of the intended recipient, and aqueous or non-aqueous sterile suspensions which may include suspending and thickening agents.

A parenteral composition may include a solution or suspension of the compounds in a vehicle such as sterile water or a parenterally acceptable oil. Alternatively, the solution can be lyophilised; the lyophilised parenteral pharmaceutical composition can be reconstituted with a suitable solvent just prior to administration.

The formulations may be presented in single dose or multi-dose containers, for example, sealed ampoules or vials, and may be stored in lyophilised condition requiring only the addition of the sterile liquid carrier, for example water for injections, immediately prior to use. Extemporaneous injection solutions and suspensions may be prepared from powders, granules, lyophilized and sterile compresses.

In the case of parenteral administration, the composition may also be provided with the active ingredients in separate containers that can be suitably admixed according to the desired dosage taking into account the weight, age, gender and health status of the patient in need thereof.

The invention also encompasses compositions or kits for the treatment of tumours as defined above comprising one or more inhibitor according to the invention and, optionally, an anticancer drug.

The kit may comprise one or more inhibitor according to the present description and an anticancer drug each in distinct vials (or the inhibitors may all be in the same vial) thus allowing a sequential or concomitant administration of the vials in the same or in different sites.

The invention also encompasses a method for the treatment and/or the prevention of a tumour comprising administering to a patient in need of an anti-tumour and/or a tumour prevention treatment, a therapeutically effective amount of at least one inhibitor according to the invention above, optionally in combination with another anti-tumour therapy.

The term therapeutic amount includes from one dose to a whole treatment regimen of the inhibitor causing a delay on the tumour growth rate, and/or a partial to total regression of the tumour and/or a loss of tumourigenic activity of tumour cells thus preventing the development of metastasis.

All the indications concerning the inhibitors (kind of inhibitors, classes of tumours treated/or prevented by the inhibitors) disclosed in the present application and in claims 1-18 appended, apply also to the composition, the kits, and the medical treatment method.

The following examples and experiments provide at least the scientific basis and modes to carry out the invention.

All the cell lines used in the experimental section, providing scientific support to the invention claimed, when non commercially available, where obtained with the informed consent of the patients.

### EXPERIMENTS AND EXAMPLES

1. Cell Cultures:

Establishment of primary BTSC (Brain Tumour Stem Cells) lines was performed as reported before by Galli, R. et al. “Isolation and characterization of tumourigenic, stem-like neural precursors from human glioblastoma” Cancer research 64, 7011-7021 (2004) and by Gritti, A. et al. “Multipotential stem cells from the adult mouse brain proliferate and self-renew in response to basic fibroblast growth factor” J Neurosci 16, 1091-1100 (1996). The BTSC clones 1, 30pt and 83, used in this work were also published in Ricci-Vitiani, L. et al. “Tumour vascularization via endothelial differentiation of glioblastoma stem-like cells” Nature 468, 824-828.

All BTSC clones were cultured in serum-free medium containing 20 l/ml glucose 30%, 15 l/ml sodium bicarbonate 7.5%, 5 l/ml Hepes 1M, 2 g/ml heparin, 4 mg/ml BSA, 10 l/ml glutamine and PS dissolved in DMEM/F12 medium and supplemented with 20 ng/ml EGF and 10 ng/ml bFGF and 100 l/ml Hormone mix 10× containing 200 l/ml DMEM F12 5×, 20 l/ml glucose 30%, 15 l/ml sodium bicarbonate 7.5%, 5 l/ml Hepes 1M, 1 mg/ml apotransferrin, 50 mg/l insulin, 96.6 mg/l putrescine, 100 l/l selenium 3×10-3M, 100 l/l progesterone 2×10-3M. Cultures were expanded by mechanical dissociation of spheres followed by re-plating of both single cells and residual small aggregates in complete fresh medium and were incubated at 37° C. with 5% CO2.

The HEK293T human renal epithelial cell line was used to produce lentivirus particles. These cells were maintained in Dulbecco's modified Eagle's medium (DMEM), supplemented with 10% fetal bovine serum (FBS) and they were incubated at 37° C., 5% CO2. These cells were expanded using trypsin (0.05%, Gibco) 1:4 diluted in PBS to detach them from the plastic. Medium containing serum was added to inactivate the action of the trypsin. After centrifuge them at 1200 rpm for 5 minutes, HEK293T cells were resuspended in fresh medium and replated in T75 flasks.

Glioblastoma cell lines such as U87MG, U251, LN215 and T98G were purchased from American Type Culture Collection and were maintained in DMEM+10% FBS (U251 and U87MG) and in RPMI+10% FBS (LN215 and T98G). Same applies for the neuroblastoma lines SHSY-S5Y (RPMI). The lines SHEP and Kelly were gifts from Prof. Dr. Simone Fulda.

The myeloma cell line X63-Ag8.653 was a gift from Dr. Martin Sprick and was maintained in RPMI+10% FBS. The hybridoma line α-ITGA7-1 was cultivated under identical conditions.

2. Differentiation of Primary BTSC Lines:

BTSC1, BTSC151, BTSC23 and BTSC28 cells were plated on to a basement membrane-like extracellular matrix extract (matrigel)-coated 6 well plate (50.000 cells/well). We used 2 ml/well of chilled (4° C.) matrigel (BD Biosciences) 1:50 diluted in DMEM F12+10% FBS and put the 6 well plates in incubator over night. When incubated at 37° C., the matrigel proteins self-assemble producing a thin film that covers the surface of the well. The day after we washed two times with PBS each well and then we plated the cells for 7 to 10 days. Then FACS analysis was performed.

3. Lentiviral Particle Production and Infection:

Lentiviral particles were produced by transient triple-plasmid transfection of HEK293T (human embryonic kidney cell) host cells. The day before transfection 8×10⁶ cells were seeded in a T160 flask. HEK293T cells were cotransfected with 28 g of the packaging vector (psPAX2), 12 g of the envelope vector (pMD2) and 40 μg DNA of interest such as pLKO.1 B10, pLKO.1 C1. The shRNA containing vectors were purchased from Thermo Scientific, and the packaging constructs were obtained from the ADDGENE consortium) For the transfection we used standard CaPO4 transfection methods, preparing the mixes according to the manufacturers recommendation (CalPhos, Clontech, Mountain View, Calif.). The medium was changed 16 hours post transfection and virus-containing supernatant was collected after 72 h and centrifuged at 1800 rpm for 5 minutes, 25° C. The supernatant was then filtered with 0.45 m filter to remove residual cells in the supernatant. The cells that have to be infected need to be centrifuged at 1800 rpm for 5 minutes to be mechanically disgregated and plated in a 6 well plate with virus-containing supernatant filtered and 10 g/l polybrene (SIGMA). The cells incubated with virus were centrifuged at 1800 rpm for 30 minutes and put in the incubator over night. One day after we washed the cells with PBS and changed the media. 48 h after the infection cells were selected with 10 g/l puromycin. All experiments were performed 10-20 days post transfection.

4. Growth Curves:

After plating equal number of cells for each time point of our growth curves, the Cell Titer-Glo® Luminescent Cell Viability Assay (Promega) was used to determine the viability of the cells in culture depending on the amount of the ATP present, which indicates the presence of metabolically active cells. Control wells containing medium without cells were prepared to obtain a value for background luminescence. After adding the reagent to the cells we mixed the content on a shaker for 10 minutes at room temperature and then we transferred it to a white-walled multiwell plate suitable for luminescence measurements using a multichannel pipette to reduce the pipetting error. The luminescent signal was recorded after 10 minutes of incubation at room temperature. We normalized the values obtained for each time point to day 0 of the same treatment Western blotting:

Protein lysates were prepared using standard RIPA buffer (150 mM NaCl; 20 mM Tris, pH 7.2; 0.05% SDS; 1.0% Triton X-100; 1% Deoxycholate; 5 mM EDTA) and the proteins were separated with NuPAGE gels, using MOPS buffer (Invitrogen). Proteins were blotted according to the manufacturers' recommendation (Invitrogen). The membranes were blocked for 1 hour with PBST (PBS, 0.02% TWEEN20) containing 5% blotting grade nonfat powdered milk.

Primary antibodies used to detect the regulators of cell cycle progression were: phospho-ChK-1 Ser 345 (Cell Signaling #2348), phospho-Rb Ser 807811 (Cell Signaling #9308), cyclin B1 (G-11): sc-166757 (Santa Cruz Biotechnology Inc.), MELK (E-14): sc-48035 (Santa Cruz Biotechnology Inc.), PLK1 rabbit mAb (Cell Signaling #4513). All these antibodies were 1:1000 diluted. The secondary antibody used were obtained from Southern Biotechnologies) and 1:10.000 diluted. After the detection we washed 3 times the membranes for 5 minutes with PBST and then we used the stripping solution (50 mM glycine buffer at pH 2.3) to inhibit HRP activity on the blot in order to detect more proteins on the same membrane. Finally we blocked non-specific binding by placing the filters in PBST containing 5% powder milk for one hour shacking. After the blocking, the membranes were incubated overnight at +4° C. with the next primary antibody diluted in PBST with 5% powdered milk.

5. Flowcytometric Analysis:

Intracellular staining for cytoplasmatic proteins such as nestin and GFAP was carried out. The cells were washed one time in PBS (4° C.) and re-suspended in 100 l PBS+100 l PFA 4% for 10 minutes at room temperature to fix them and were washed 2 times in PBS/Triton 0.1% to permeabilise cell membranes. The primary antibody was diluted in PBS/Triton 0.1% and the cells were stained 45 minutes on ice. After two washes in PBS/Triton 0.1% at 4° C., we diluted the secondary antibody in PBS/Triton 0.1% and the cells were stained 30 minutes on ice (dark) to preserve the fluorescence of the dye used to label the secondary antibody. One wash in PBS/Triton 0.1% (4° C.) and another wash in PBS/BSA 0.5% (4° C.) were carried out. Finally, the cells were resuspended in 100 l PBS BSA 0.5% in FACS tubes for the flow cytometric analysis. We performed the extracellular staining to evaluate surface markers such as CD133 and to re-screen the supernatants of hybridomas against BTSC1 and towards differentiated glioblastoma cell lines. The cells were washed one time in PBS BSA 0.5% (4° C.) and then the primary antibody was diluted in PBS BSA 0.5% or the hybridoma SN was directly used to incubate the cells 45 minutes on ice. Cells were then washed twice in PBS/BSA 0.5% and incubated with the secondary antibody in PBS BSA 0.5%. The cells were stained for 30 minutes on ice (dark) and then were washed two times in PBS/BSA 0.5% to be resuspended in 100 l PBS BSA 0.5% containing 5 g/ml 7-Amino-actinomycin D (7-AAD) in 5 ml FACS tubes for the analysis. 7-AAD intercalates into double-stranded nucleic acids and is excluded by viable cells.

6. Animal Experiments:

For all in vivo studies heavily immunocompromised NSG mice (NOD scid gamma or NOD. Cg-Prkdc^{scid} ll2^{tm1Wjl}/SzJ) from Jackson labs were used. For subcutaneous xenograft models, 1×10⁶ BTSC1 or BTSC83 cells transduced with lentiviral vectors for the downmodulation of ITGA7 expression or 1×10⁵ sorted SHSY-S5Y cells were injected into the flanks of the mice (n=12 group). Tumour growth was monitored and tumour size was measured with a caliper. For the orthotopic model, 50.000 BTSC30pt cells virally transduced with a luciferase expression construct (pTWEEN-LUC) and the shRNA constructs indicated were injected in 3 μl DMEM/F12 using a small animal stereotactic device. Tumour growth was monitored 3-5 months after injection by in vivo bioluminescence methods: In short, mice were injected with 150 mg/kg Luciferin i.p. After 15 min the bioluminescence was detected with a Xenogen IVIS 100 small animal in vivo imaging system.

7. Hybridoma Generation:

Mice immunised with antigen were euthanized, the spleen was removed under aseptic conditions and the splenocytes were fused with X63-Ag8.653 myeloma cells as described in the attached “Cell Fusion/Hybridoma Production Protocol”. The hybridomas were selected with HAT supplement and one day after fusion the cells were directly plated in 96 well plates to densities of 1-5 surviving hybridoma clones/well (9000 well/complete spleen). After 10 days of HAT selection the hybridoma supernatants were tested for immunoreactivity towards primary brain tumour stem cells as described in “high throughput hybridoma screening”. Promising hybridomas were 2 times subcloned by single cell laydown using a FACS Aria flowcytometer and immunoreactivity was confirmed by flowcytometric analysis of antibody surface binding on BTSC1. The hybridomas were frozen and stocked in nitrogen. Antibody purification using a ReSURE protein A column (Amersham Pharmacia) was performed according to the manufacturers' recommendations.

8. High Throughput Hybridoma Screening:

In order to test big numbers of hybridoma supernatant for specific reactivity towards surface molecules on primary brain tumour stem cell like cells, a flowcytometry based high throughput screening method was set up. To this end, purified peripheral blood lymphocytes (PBLCs) were labelled for 20 min with 10 μg/ml HOECHST 33342 at 37° C. No-incorporated dye was removed by 3 washes with PBS. The cells were counted and mixed in a 3:1 ratio with a single cell suspension of non-labelled BTSC1 cells. The staining was performed in 96 well round bottom plates and 20000 cells well were incubated with 80 μl of the individual hybridoma supernatants for 1 hour on ice. The supernatants were removed and cells were washed 2 times with PBS+0.5% BSA. The bound antibody was then labelled with an PE-conjugated, mouse Ig-detecting secondary antibody (Invitrogen). The secondary antibody was used 1:200 diluted in PBS+0.5% and incubated for 30 min on ice. The cells were again washed and FACS analysis was performed using a FACS LSR II flowcytometer equipped with a HTS-96 plate holder. This methodology allowed for the analysis of more than 600 supernatants per day. The hybridomas producing antibodies, which bound selectively to BTSC1 but not to PBLCs were further analysed for binding on other BTSC or differentiated lines as explained above.

9. Identification of the Anti-ITGA7 Specificity of the Antibody α-ITGA7

The antibody herein named α-ITGA7 showing a very strong immune reactivity to all BTSC lines (FIG. 1), while only weak positivity to the conventional cell lines tested (FIG. 1) was selected. The antibody showed a preferential binding on the surface of undifferentiated cells (FIG. 2) by flowcytometric analysis.

The strong binding of the antibody produced by one selected hybridoma to BTSC1 (see FIGS. 1 and 2) was demonstrated to be an ITGA7 specific binding by immunoprecipitation followed by mass spectrometric analysis. The molecular weight of the antigen was determined by performing a cell surface biotinylation prior to cell lysis and precipitation using the α-ITGA7 antibody. By western blot analysis a strong signal was detected at a molecular weight of approx. 100-110 kDa (FIG. 3a). Subsequently a protein signal in the silver gel and even in the coomassie stained preparative SDS-PAGE gel (FIG. 3b, c) were detected. The bands from the gel were excised, the contained protein was digested with trypsin and a tandem mass spectrometric analysis was carried out. The weight corresponded to ITGA7.

The human ITGA7-encoding cDNA was cloned in a lentiviral expression vector (pTWEEN) and the protein was expressed in HEK293T cells, which do not express endogenous ITGA7 on their surface. The ITGA7 expressing HEK293T cells were assayed for binding with α-ITGA7 antibody, which correlated with the positivity for the co-expressed GFP (FIG. 4a). In order to rule out the possibility of an overexpression artifact, the expression of ITGA7 in BTSC1 by stable transduction with lentiviral shRNA constructs was down regulated. In contrast with the cells treated with control shRNA, which are strongly positive for antibody binding, the binding of the antibody is reduced in cells treated with ITGA7 shRNA (FIG. 4b). Taken together, these results proof that α-ITGA7 antibody indeed recognizes ITGA7.

10. Statistics Analysis:

All experiments were repeated at least three times. All numerical data were described as mean±SEM. Data was analysed using the two-tailed student t-test. A probability value of 0.05 or less was considered significant.

11. ITGA7 Positive SH-SY5Y (Neuroblastoma Cells) are More Tumourigenic Compared to ITGA7 Negative Cells

To determine if there is any functional relevance of ITGA7 as marker for highly aggressive brain tumour cells, a panel of conventional tumour cell lines was tested for the expression of this antigen. It was found, that the neuroblastoma line SH-SY5Y contains a small subpopulation (10-15%) of cells strongly positive for ITGA7 (FIG. 5a). These cells were sorted by flowcytometry and were found to be growing significantly faster in vitro, when compared to the ITGA7 negative sorted cells (FIG. 5b). Interestingly, the cells positive sorted lost the expression of ITGA7 over time, indicating, that only the fast proliferating subpopulation of SH-SY5Y is positive for this protein (FIG. 5a). Importantly, the enhanced aggressiveness of ITGA7 positive SH-SY5Y in vivo was also verified. Cells positive for ITGA7 engrafted significantly faster in immune-deficient NSG mice, when compared to cells negative for this protein (FIG. 5c). Hence, these data strongly support that ITGA7 expressing cells represent an aggressive subpopulation of SH-SY5Y cells.

12. ITGA7 Positive Primary BTSCs are Enriched in Clonogenic Cells

Having detected a higher level of aggressiveness in the ITGA7 positive population of SHSY-S5Y cells, ITGA7 was also tested as a marker for the aggressiveness of primary BTSCs. For this reason three different BTSC lines we selected (BTSC30pt, BTSC83TW and GBM T1) in order to test cells expressing high versus cells expressing low levels of ITGA7 by flow cytometry. The cells were directly sorted into 96 well plates to 1 and 3 cells/well and their clonogenic capacity was determined. The fraction of cells with high ITGA7 expression displayed a significantly higher clonogenic capacity in all three lines investigated (see FIG. 6). These results indicate an enrichment of potentially tumour initiating cells in the ITGA7 positive population of tumour stem like cells.

13. Genetic Interference with ITGA7 Impairs Proliferation and Clonogenic Survival of BTSC1 in Vitro

As the data obtained strongly supported that the presence of ITGA7 on the cell surface is directly linked to tumour cell growth, it has been verified if interfering with the expression of the ITGA7 molecule leads to a reduction in tumour cell growth. In order to test this ITGA7 expression was inhibited by infecting BTSC1 cells with lentiviral particles encoding for small hairpin (sh)RNA, targeting the cDNA of human ITGA7. FACS analysis confirmed a substantial knockdown of ITGA7 protein (by binding with an anti-ITGA7 antibody) by approx. 90% in cells treated with the shRNA, targeting the cDNA of human ITGA7, compared to the cells treated with control shRNA. These results could be obtained without any prior selection, thus reducing potential artifacts due to selection antibiotics. Cell proliferation and clonogenicity assays with BTSC1 and BTSC83 cells knocked down for ITGA7 were carried out and a highly significant decrease in cell growth in cells with reduced ITGA7 levels was detected (FIGS. 7a and 8c). Even more striking was the reduction of the clonogenic survival of the cells treated with ITGA7 shRNA, which was almost completely blunted, while up to 30% of the cells transduced with control shRNA were clonogenic (FIGS. 7b and 7d). To rule out cell type specific effects and shRNA artifacts, the ITGA7 knockdown was repeated with 2 additional primary BTSC lines (BTSC 30pt, BTSC151) and GBM T1, a very low passage (P3-5) spheroid culture from freshly isolated gliomablastoma cells. As shown in FIG. 7e-f all the lines show a significant reduction in proliferation, when treated with ITGA7 targeting shRNA constructs. Taken together these results clearly underline an essential role for ITGA7 for proliferation of tumour stem cells in vitro, which seems to be entirely independent from the cellular system used.

14. ITGA7 is Crucial for Proliferation and Clonogenic Survival of BTSC1 in Preclinical Animal Models

Having confirmed the anti-proliferative effect of neutralizing ITGA7 in vitro, the system was transferred in preclinical in vivo model systems. The results obtained show that in severely immune deficient mice (NSG mice) the cells knocked down for ITGA7 expression are significantly less tumourigenic when compared with the cells transduced with the control shRNA construct in a subcutaneous tumour model (FIG. 9). The results were very evident using BTSC1 cells, which show a high ITGA7 expression, as model system. These cells formed perceptible tumour nodules significantly later, when ITGA7 expression was suppressed (FIG. 9a). More importantly only one out of 6 mice showed limited tumour growth 5 months after engraftment. In contrast the control cells readily formed small nodules and developed big tumours with 100% efficiency (FIG. 9b). Also using BTSC83 cells, which display a lower surface expression of ITGA7 but still display ITGA7 surface expression, a significant delay in engraftment was also detected, even though 100% of the mice injected developed tumours (FIG. 9c). However, the tumour growth of cells in which ITGA7 expression was suppressed by shRNA was significantly slower, when compared with the control cells (FIG. 9d). The same was true for the weights of the tumours isolated at the end of the experiment (data not shown).

An orthotopic GBM model by intracranial injection of luciferase transduced BTSC30pt was hence set up. Also in this case the cells were transduced with lentiviral vectors encoding either for a non-silencing control shRNA (ctr) or for ITGA7 targeting shRNAs. After viral transduction the cells were recovered for 7 days and then 30 000 cells/mouse were injected using a stereotactic device. The tumour engraftment and growth was measured 90 days after the initial engraftment by using an IVIS small animal imaging system. As shown in FIG. 10, 4 out of 5 mice engrafted with cells transduced with the ctr shRNA construct showed strong bioluminescence produced by the luciferase of the injected cells. The bioluminescence of BTSC30pt transduced with ITGA7 shRNA of SEQ ID NO 1 was significantly less intense (approximately factor 10 less). In the third group of animals the difference was even more pronounced, as 4 out of 5 mice injected with cells transduced with ITGA7 shRNA of SEQ ID NO 2 showed no successful engraftment of luciferase positive cells. These results confirm the data of the heterotopic model system and indicate a central role of ITGA7 in the tumourgenicity of tumour stem cells in preclinical in vivo models.

15. Anti Integrin Alpha7 Antibody Suppresses Tumour Growth in Vivo

Having identified ITGA7 as a tumour promoting surface molecule, the inventors performed an in vivo experiment to block the interaction of integrin alpha7beta1 with the tumour microenvironment. Treatment of the mice with two weekly doses of 10 mg/kg anti integrin alpha 7 antibody (see FIG. 12a) led to a significant reduction of tumour engraftment in an heterotopic xenograft model based on BTSC1 cells. While shortly after implantation the luciferase activity was equal with the control group (see FIG. 12b), 18 days post implantation the tumour engraftment was significantly impaired in the mice treated with the integrin alpha 7 antibody (FIG. 12c). These results indicate a potential therapeutic value of antibody mediated integrin alpha7 targeting in vivo.

As pointed out before, the antibody treatment was repeated two times/weekly for a total of 4 weeks. At the indicated time points tumour engraftment was determined by detection of luciferase activity using an IVIS Xenogen 100 small animal in vivo imaging system.

The identical experiment was repeated with an independent BTSC line (BTSC-T3).

The final data obtained are reported in FIGS. 12d) and e); *** and * indicate a p value of <0.05 and <0.001, respectively (two tailed student t test).

16. Possible Mechanism of Action, ITGA7 Mediates Cell Cycle Progression in Primary Brain Tumour Stem Cells

Without being bound to theories, having confirmed the inhibition of proliferation in ITGA7 deficient cells, the inventors also investigated the possible molecular mechanism at the basis of this observation. For this reason western blot analysis and probing for proteins implicated in cell cycle regulation was carried out. In both BTSC lines investigated, cells knocked down for ITGA7 expression showed a marked decrease in phosphorylated retinoblastoma (RB) protein, Cyclin B1 and Polo like kinase 1 (PLK1) (FIG. 8a). The results suggested a block in the cell cycle progression. The biochemical evidence for a block in cell cycle progression was also confirmed by flowcytometric cell cycle analysis, showing for both lines an accumulation of nuclei in G1 phase (FIG. 8b-d). As for the biochemical analysis, this increase of cells in G1 phase could be observed for two independent shRNA constructs, ruling out any shRNA specific off-target effects. These results suggest, that ITGA7 is crucial for the aberrant cycle progression of primary tumour stem like cells.

17. Antibody and shRNA Mediated Functional Blocking of ITGA7 Function Impedes Laminin Signaling and Invasion of GBM Cells in Vitro

By performing IF analysis it was found laminin strongly expressed in patient derived GBM samples (FIG. 14a). As it was demonstrated that ITGA7 mediates the locomotion of skeletal myoblasts on a laminin substrate, a test was carried out in order to asses whether ITGA7 might be a mediator of cell spread and invasion of the herein described spheroid GBM cells in vitro. To quantify the invasive potential of the cells, an invasion assay was carried out using laminin coated transwells and a highly significant reduction of invasion in cells knocked down for ITGA7 was detected (FIG. 14b). To understand the biological consequences of ITGA7 ablation in more detail, the intracellular pathways involved in integrin signaling were investigated. Focal adhesion kinase (FAK) and Src were readily phosphorylated at Tyr397 and Tyr416, respectively, when BTSCs were seeded on laminin coated plates. This effect was completely blunted by the knockdown of ITGA7 using two shRNA constructs in different BTSC lines (FIG. 14c). Having identified its potential role in GBM migration and proliferation, it was investigated whether it is possible to therapeutically interfere with ITGA7 function. To test this hypothesis in quantitative manner, the invasion assay was repeated with different BTSCs and a strong inhibition of the invasive phenotype by both the intact antibody and the F(ab) fragment generated from anti-ITGA7 was found (FIG. 14d) Importantly, the anti-ITGA7 was capable to block the outside-in signaling of laminin. As described for the ITGA7 knockdown, also two independent GBM stem cell lines pre-treated with anti-ITGA7 showed a massive reduction in laminin induced FAK and Src phosphorylation when compared to non treated cells or cells pretreated with an isotype control antibody (FIG. 14e). These results underline a blocking function of anti-ITGA7 with regards to cell invasion and laminin induced signaling.

18. Anti-ITGA7 Significantly Reduces the Growth and Invasion of BTSC in Intracerebral Xenografts

Intracerebral injection of BTSCs in immunocompromised mice generates highly infiltrative tumour xenografts that closely mimic the behaviour of malignant gliomas. Within a few weeks after grafting, BTSCs colonize the injection site and spread towards distant brain regions with a special tropism for the large paths of white matter, like the corpus callosum and anterior commissure. Brain xenografts of BTSCs were used as an experimental model system to assess whether anti-ITGA7 exerts its anti-tumour effect in the in vivo condition. First, stable luciferase-expressing BTSC1 cells in immunodeficient mice were intracranially engrafted and the tumour growth was measured by bioluminescence. As shown for the subcutaneous model, anti ITGA7 treatment also slowed down the tumour growth in the orthotopic model (Fig. FIGS. 15a and 15b). In order to investigate the migratory behaviour of the cells in more detail, stable GFP-expressing BTSC1 were grafted into the striatum of SCID mice. 3 μg anti-ITGA7 or isotype control (IgG2a) were administered locally at the time of cell injection and over the following 4 weeks by systemic injection. By 8 weeks after grafting, control mice harboured tumours that invaded the homolateral striatum, piriform cortex, corpus callosum, anterior commissure, internal capsule, optic tract, septal nuclei, and fimbria-hyppocampus, whereas the degree of brain invasion was significantly reduced in anti-ITGA7 treated mice (FIG. 15c). The volume of the brain region invaded by GFP+BTSCs was 4.22±0.71 (mean±sem) and 11.02±1.29 mm3 in anti-ITGA7 treated mice and in control mice, respectively (p<0.005; FIG. 15d). Treatment with anti-ITGA7 dramatically lowered the number of tumour cells that infiltrated the corpus callosum, which scored 8.75±2.02 (mean±sem) and 80.25±8.49 cells in anti-ITGA7 treated mice and in controls, respectively (p<0.001; FIG. 15e). The number of cells in the anterior commissure was also significantly lower in anti-ITGA7 treated animals compared to control ones (143.5±18.12 and 17.75±4.31 cells, respectively; p<0.001). At the brain site where anti-ITGA7 was injected, we did not observe pathological changes of the brain parenchyma suggesting chronic inflammation or toxicity.

19. ITGA7 is Expressed by Melanoma Cells and Antibody Mediated Interference with ITGA7 Function Significantly Reduces Melanoma Cell Invasion

To investigate the expression of ITGA7 protein in other cellular systems, a surface staining and flowcytometric analysis of 10 independent melanoma cell lines was carried out. It was found that ITGA7 expressed on the cell surface by 14.3 to almost 100% of the melanoma cells depending on the cell line used (FIG. 16a). The expression of ITGA7 could be verified by western blot, which like the flowcytometric analysis showed the highest expression of ITGA7 in SK-Mel 30, Preyer and MeWo (FIG. 16b). It was then investigated it the ITGA7 function could be blocked in the melanoma system with an anti-ITGA7 antibody a significant reduction (max 85% for SK-Mel 30 cells) of cell invasion into the laminin matrix in the presence of 1.4A12 anti ITGA7 antibody was observed (FIG. 16c). These data strongly suggest a similar function of ITGA7 in melanoma as compared to glioblastoma multiforme.

ITGA7 is Expressed by Ovarian Cancer, Chondrosarcoma and Osteosarcoma Cells and Antibody Mediated Interference with ITGA7 Function Significantly Reduces Melanoma Cell Invasion

The surface expression of ITGA7 protein on ovarian cancer cells and primary sarcoma derived stem cell like lines was also investigated. A surface staining and flowcytometric analysis of 2 ovarian cancer cell lines was carried out. ITGA7 was expressed by both lines investigated by 12.9% and 84.3% of the cells for SK-OV3 and A2780, respectively (FIG. 17a). Low passage primary sarcoma stem like cells for the surface expression of ITGA7 was also tested and ITGA7 was detected on the surface of the vast majority of the cells. 93.8% and 98.8% of Osteosarcoma and Chondrosarcoma derived cancer stem like cells were positive for ITGA7 (FIG. 17b). The data obtained supports that also these tumours are potential targets for anti ITGA7 therapy.

1. A method for treatment and/or prevention of a tumour comprising administering to a patient in need thereof, a therapeutically effective amount of at least one inhibitor of integrin alpha 7 activity.

2. The method according to claim 1, wherein said tumour expresses the ITGA7 protein or the ITGA7/ITGB1 dimer on the surface of at least a population of tumour cells.

3. The method according to claim 2, wherein said tumour cells are tumour stem-like cells.

4. The method according to claim 2, wherein said tumour is selected from the group consisting of CNS tumours, primary brain tumours, lower grade brain tumours, Head/Neck (Oral, Nasopharyngeal) tumours, digestive system tumours, respiratory system tumours, bone tumours, skin tumours, blood tumours, urogenital tumours, nervous system tumours, endocrine system tumours, sarcomas, and gynaecological cancers.

5. The method according to claim 4, wherein said tumour is a tumour of neuroectodermal origin, a mesenchymal tumour, an epithelial tumour, or a tumour showing a epithelial-mesenchymal transition.

6. The method of claim 4, wherein said tumour is selected from the group consisting of glioblastoma multiforme, astrocytoma grade IV, glioma, astrocytomas grade I-III, cerebral meningiomas, pituitary adenomas, non-small cell lung cancer (NSCLC), lung adenocarcinoma, lung squamous cell carcinoma, lung large cell carcinoma, colon carcinoma, breast carcinoma, all types of melanoma, all types of ovarian cancer and sarcomas, Ewing's sarcoma, osteosarcoma, soft-tissue sarcomas, leiomyosarcoma, and rhabdomyosarcoma.

7. The method according to claim 1 wherein said inhibitor inhibits expression of the gene encoding integrin alpha 7.

8. The method according to claim 7, wherein said inhibition of expression of the gene encoding integrin alpha 7 is exerted at the transcriptional level or the translational level.

9. The method according to claim 8, wherein said expression is inhibited by RNA interference.

10. The method according to claim 9, wherein said inhibitor is an RNA-based inhibitor selected from the group consisting of shRNA, siRNA, and miRNA.

11. The method according to claim 10, wherein said RNA-based inhibitor comprises LNA nucleotides.

12. The method according to claim 10, wherein said RNA-based inhibitor is a cholesterol-siRNA conjugate.

13. The method according to claim 10, wherein said RNA-based inhibitor is inserted in an adeniviral, lentiviral, or retroviral vector.

14. The method according to claim 1, wherein said inhibitor inhibits activity of the ITGA7 protein or of the ITGA7/ITGB1 dimer.

15. The method according to claim 14, wherein said activity is inhibited by interaction between the inhibitor and the ITGA7 protein or the ITGA7/ITGB1 dimer on the cell surface.

16. The method according to claim 15, wherein said inhibitor is an antagonist of the ITGA7 protein or the ITGA7/ITGA1 dimer.

17. The method according to claim 14, wherein said inhibitor is an antibody, a Fab, a F(ab′)2 fragment, a single chain antibody, a partially or fully humanized, or a recombinant human antibody.

18. The method according to claim 14, wherein said inhibitor disrupts interaction between the ITGA7 and the ITGB1 subunits.

19. The method according to claim 14, wherein said inhibitor blocks activation of focal adhesion kinase (FAK) and the ITGA7/ITGB1 dimer.

20. A pharmaceutical composition comprising one or more inhibitors of integrin alpha 7 activity and a pharmaceutically acceptable carrier for treatment and/or prevention of tumours.
